(19) United States
(12) Patent Application Publication
(10) Pub. No.: US 2009/0035287 A1
(43) Pub. Date: Feb. 5, 2009

(54) ATRIAL NATRIURETIC PEPTIDE (ANP) SPLICE VARIANTS AND METHODS OF USING SAME

(76) Inventors: Zurit Levine, Herzliya (IL); Michal Ayalon-Soffer, Ramat-HaSharon (IL); Merav Beiman, Ness Ziona (IL); Ronen Shemesh, Modiin (IL)

Correspondence Address: FENNEMORE CRAIG, 3003 NORTH CENTRAL AVENUE, SUITE 2600, PHOENIX, AZ, 85012, US

(21) Appl. No.: 12/088,132

(22) PCT Filed: Sep. 26, 2006
(86) PCT No.: PCT/IL2006/001123

§ 371 Date: May 13, 2008

Related U.S. Application Data
(60) Provisional application No. 60/719,969, filed on Sep. 26, 2005.
(60) Provisional application No. 60/779,407, filed on Mar. 7, 2006.

(51) Int. Cl. A61K 35/12; C12N 15/11; C07K 14/00; C12N 15/00; A61K 38/00; A61P 9/00; C12N 5/06; C12P 21/04; A61K 31/7088

(52) U.S. Cl. 424/93.21; 536/23.5; 530/350; 530/326; 435/320.1; 435/325; 435/69.1; 514/44; 514/12

(57) ABSTRACT

Novel ANP polypeptides and polynucleotides encoding same are provided. Also provided are methods and pharmaceutical compositions which can be used to treat ANP-related diseases and disorders including cardiovascular disorders using the polypeptides and polynucleotides of the present invention.

### FIELD OF THE INVENTION

The present invention relates to Atrial Natriuretic Peptide (ANP) splice variant polypeptides, polynucleotides encoding these variant polypeptides, vectors comprising the polynucleotides and host cells comprising same, and more particularly, to therapeutic compositions and use thereof.

### BACKGROUND OF THE INVENTION

Atrial Natriuretic Peptide (ANP), also called atrial natriuretic factor (ANF), is a 28-amino acid polypeptide, that belongs to the mammalian natriuretic peptide family of peptides comprising ANP (atrial natriuretic peptide), BNP (brain natriuretic peptide) and CNP (C-type natriuretic peptide). ANP and BNP are endocrine cardiac hormones that decrease blood pressure through the stimulation of renal sodium and water excretion, vasorelaxation, and antagonism of the renin-angiotensin-aldosterone system. CNP signals in a paracrine fashion to stimulate vasorelaxation and long bone growth. A new natriuretic peptide, Dendroaspis natriuretic peptide (DNP), homologous to the venom of Dendroaspis augusticeps, the Green Mamba snake, has recently been discovered in human plasma and atrial myocardium and its therapeutic activity is being investigated (Lisy O, et al. Hypertension. 2001 37(4):1089-94).

ANP is mainly stored in heart atrial cells as a 126 amino acid prohormone corresponding to SEQ ID NO:13 (ANF_HUMAN) without a signal peptide (amino acids 1-15 of SEQ ID NO:13). The prohormone contains 4 hormone peptides: long-acting natriuretic peptide (consisting of the first 30 amino acids: 1-30), vessel dilator (amino acids 31-67), kaliuretic peptide (79-98) and ANP (amino acids 99-126), all of which regulate blood pressure and maintain plasma volume. ANP is cleaved by the serine-protease corin and is secreted from the atrial cells in response to increased pressure and stretch (Kangawa et al., Biochem Biophys Res Commun. 1984, 121(2):585-91). ANP is capable of rapidly lowering blood pressure through a general relaxation of veins, increased capillary filtration and decreasing Na⁺/water re-absorption. ANP is unique in its high affinity to its receptors, but it is quickly degraded with a half-life of about 3 minutes. Urodilatin is a known alternative cleavage product, 32-amino acids long, found in kidney. It is derived from the ANP precursor, (amino acids 95-126) and comprises ANP with four additional amino acids at the N-terminus that make it resistant to neutral endo-peptidase degradation, and hence urodilatin is more active in-vivo than ANP, due to longer half-life (Saxenhofer et al., Am J. Physiol. 1990 259(5 Pt 2):F832-8). All the natriuretic peptide family members have a conserved central core with two disulphide linked cysteine residues, found to have a critical role in stabilizing the loop structure of the active peptide. Cleavage of known ANP C-terminal arginines that are coded for by the human ANP T2332C allele, as well as in most mammalian ANPs, was suggested by Zivin et al., (Zivin R A, et al., Proc Natl Acad Sci USA. 1984 81(20):6325-9). These residues have never been observed among the numerous mature peptides reported to display ANP activity, suggesting COOH-terminal processing of the peptide resulting from this allele.

Truncating the ANP protein just before the C-terminal tyrosine was suggested to have a positive effect on ANP activity (Scarborough et al., J Biol Chem. 1986 261(28):12960-4), with the truncated protein showing a two-to-three-fold increase in receptor activation.

The known effects of natriuretic peptides are mediated through two cell-surface guanylyl cyclase receptors: Natriuretic peptide receptors A (NPR-A/GC-A) and B (NPR-B/GC-B) (Potter and Hunter, 2001; The Journal of Biol. Chem., 276:6057-6060). These are members of the transmembrane guanylyl cyclase family that mediate the effects of natriuretic peptides via the second messenger, cGMP. Both receptors consist of an extracellular ligand-binding domain, a single membrane-spanning region, and intracellular kinase homology, dimerization, and carboxyl-terminal guanylyl cyclase domains. In addition, all three natriuretic peptides bind to a third protein called the natriuretic peptide clearance receptor (NPR-C), which lacks enzymatic activity and functions to remove natriuretic peptides from the circulation via receptor mediated, but ligand-independent, internalization and degradation (Potter and Hunter, 2001; The Journal of Biol. Chem., 276:6057-6060).

The Natriuretic Peptide system is the main endogenous system to counter-regulate the deleterious chronic activation of vasopressin, the Renin-Angiotensin-Aldosterone and the sympathetic nervous system, which is found in cardiovascular diseases. Reversing this imbalance has been considered a promising therapeutic approach in the treatment of Congestive Heart Failure (CHF).

U.S. Pat. No. 6,514,939 discloses methods using mutant ANF proteins, fragments, analogs, derivatives and homologs of mutant ANF proteins, the nucleic acids encoding these mutant ANF proteins, and modulators of ANF for treating or preventing ischemic diseases, in particular ischemic stroke. It particularly discloses methods of diagnosis, prognosis and screening for a disposition to diseases and disorders associated with increased levels of ANF.

WO05069724 and PCT/IL2006/000676 to the applicant of the present invention disclose ANP variant polynucleotides and their respective encoded polypeptides, for diagnostic utilities of cardiac disease, pathology, conditions and disorders comprising one or more of Myocardial infarct, acute coronary syndrome, angina pectoris (stable and unstable), cardiomyopathy, myocarditis, congestive heart failure or any type of heart failure, the detection of reinfarction, the detection of success of thrombolytic therapy after Myocardial infarct, Myocardial infarct after surgery, assessing the size of infarct in Myocardial infarct, the differential diagnosis of heart related conditions from lung related conditions (as pulmonary embolism), the differential diagnosis of Dyspnea, and cardiac valves related conditions. PCT/IL2006/000676 particularly discloses a variant of atrial natriuretic factor precursor protein, designated HUMCDDANF_{—}1_P4, having an amino acid sequence as set forth in SEQ ID NO:15, and polynucleotides encoding same.

In heart failure patients various clinical studies with variable results were reported for different ANP agonists (including Urodilatin and other ANP variants; Schmitt et al., Clin Sci (Lond). 2003 105(2):141-60). Currently, only one of the natriuretic peptides is available commercially for cardiac disease in the US, namely, BNP (Nesiritide/Natrecor, SCIOS, Sunnyvale, Calif., USA). Nesiritide (BNP) is currently used via a 3-8 h infusion (0.03 mg/kg per min) for persons with acute, decompensated heart failure. Further evaluation has revealed that there is no significant natriuresis or diuresis with nesiritide (BNP) infusion in humans with CHF. The natriuretic and diuretic effects of BNP in persons with CHF are markedly blunted compared with healthy individuals. In addition to the hypotension caused by nesiritide (i.e. BNP) in persons with CHF, there is a recent study suggesting that nesiritide significantly increases the risk of worsening renal function in persons with acute decompensated CHF. (Vesely D. Clinical and Experimental Pharmacology and Physiology 2006 33:169-176). Atrial natriuretic peptide (ANP) is commercially available in Japan under the brand name Carperitide® (Astellas Pharma, Fujisawa, Japan) and is in Phase II studies in the US. Although ANP causes natriuresis and diuresis in healthy humans, it has markedly attenuated natriuretic response in humans with CHF and in animal models of CHF. High-dose administration of ANP produces little or no diuresis in humans with CHF. Thus ANP, like BNP, has significantly blunted natriuretic and diuretic effects in persons with CHF compared with healthy subjects. Like BNP, ANP has haemodynamic effects in persons with CHF and can cause severe hypotension. Ularitide, a synthetic form of Urodilatin, is also under clinical development (CardioPep Pharma—Phase II in Europe completed).

The background art does not teach or suggest variants of ANP proteins that are useful as therapeutic proteins or peptides for a range of ANP-related clinical conditions and/or variant ANP-treatable diseases with improved properties or characteristics.

The present invention overcomes these deficiencies of the background art by providing therapeutic ANP variant proteins and peptides derived therefrom, which may be used as therapeutic proteins or peptides.

### SUMMARY OF THE INVENTION

The present invention provides novel splice-variants of ANP and fragments derived therefrom. Specifically, the present invention provides ANP-splice variants and derivatives which are useful as therapeutic peptides for diseases including but not limited to ANP-related diseases.

The present invention is of novel ANP variant polypeptides and polynucleotides encoding same, which can be used for the treatment or prevention of a wide range of diseases. The present invention further encompasses pharmaceutical compositions comprising the splice variants, vectors comprising the polynucleotides encoding the splice variants, and host cells comprising such vectors.

According to alternative embodiments the present invention further discloses derivatives of the novel ANP variants, including but not limited to cleavage, glycosylation and/or phosphorylation derivatives, as well as fusion proteins and/or chemical modifications. According to some embodiments the modification is addition of a C-terminal His/StrepII tag.

According to certain embodiments, these therapeutic variant proteins and peptides of the present invention can be modified to form synthetically modified variants according to the present invention wherein modified variants include but are not limited to fusion proteins and chemical modifications. According to certain embodiments, the fusion proteins comprise an Fc fragment. According to other embodiments the chemically modified proteins are pegylated.

Preferably, these therapeutic proteins and polypeptides derived therefrom are useful as therapeutic proteins or polypeptides for diseases wherein ANP is involved in the etiology or pathogenesis of the disease process, as will be explained in detail hereinbelow.

According to one aspect, the present invention discloses ANP variant polypeptides with a unique, novel C-terminus. Unexpectedly, it was found that replacing the C-terminal Tyr of the ANP protein by a unique amino acid tail forms an active ANP variant, which is an agonist for ANP function.

According to one aspect, the present invention provides a variant of atrial natriuretic factor precursor protein HUMCDDANF_{—}1_P5 (Variant 1) having an amino acid sequence as set forth in SEQ ID NO:16.

According to one embodiment, the present invention provides a cleavage product of HUMCDDANF_{—}1_P5 (Variant 1), selected from the group consisting of SEQ ID NO:21 to SEQ ID NO:24, denoted as peptides 1A to 1D, respectively.

According to certain preferred embodiments the ANP variant of the invention, denoted ANP-45 represents a splice variant that is encoded by exons 1 and 2 of the ANP gene (NM_{—}006172 [gi:23510318], SEQ ID NO:41), while utilizing an alternative third exon, generating a polypeptide containing a unique novel C-terminus, wherein the last C-terminal Tyr of the wild type ANP is replaced by a unique tail of 18 amino acids. The mature secretory variant ANP-45 has 45 amino acid residues in total, as set forth in SEQ ID NO:22.

According to another aspect, the present invention provides a cleavage product of the variant of atrial natriuretic factor precursor protein HUMCDDANF_{—}1_P4 disclosed in PCT/IL2006/000676 to the Applicant of the present invention (Variant 2, having the amino acid sequence as set forth in SEQ ID NO:15), selected from the group consisting of SEQ ID NO:25 to SEQ ID NO:28, denoted as peptides 2A to 2D, respectively.

It is to be understood explicitly that the term “cleavage product” encompasses any peptide comprising the amino acid sequences disclosed herein, whether derived from ANP-variant, from a protein other than ANP-variant, synthetically synthesized or produced by recombinant technology.

According to certain preferred embodiments the ANP variant of the invention, denoted ANP-79 represents a splice variant that is encoded by exons 1, 2 and 3 of the ANP gene (NM_{—}006172 [gi:23510318], SEQ ID NO:41) with an intron retention between exon 2 and exon 3 (referred herein as exon 2b), encoding a protein with a novel C-terminus. In this variant, the last C-terminal Tyr of the wild type ANP is replaced by a unique tail of 52 amino acids. The mature secretory variant ANP-79 has 79 amino acid residues in total, as set forth in SEQ ID NO:26.

According to other preferred embodiments, both new ANP variants of the present invention ANP-45 and ANP-79 contain a modified N-terminal sequence, having additional 4 amino acid residues upstream, as was described for urodilatin. The N-terminally modified ANP variants of the present invention, denoted as ANP-49 and ANP-83, have 49 and 83 amino acids residues in total, as set forth in SEQ ID NO:21 and SEQ ID NO:25, respectively.

According to certain embodiments, the ANP protein variants and derived peptides of the present invention can be modified to form synthetically modified variants according to the present invention, wherein modified variants include but are not limited to fusion proteins (including but not limited to fusion with an Fc fragment of Ig) and/or chemical modifications, including but not limited to pegylation. According to other optional embodiments, the ANP-79 and ANP-83 variants of the present invention are fused to Fc fragment, and comprise the sequence as set forth in SEQ ID NOs:37 and 38, respectively.

According to yet another aspect, the present invention provides cleavage products of a variant of the atrial natriuretic factor precursor protein HUMCDDANF_{—}1_P4 (Variant 2, SEQ ID NO:15), selected from the group consisting of SEQ ID NO:29 to SEQ ID NO:32, denoted as peptides 3A to 3D, respectively, and SEQ ID NO:33 to SEQ ID NO:36, denoted as peptides 4A to 4D, respectively.

According to another aspect, the present invention provides an isolated polynucleotide designated HUMCDDANF_{—}1_T4, encoding a novel splice variant of ANP having an amino acid sequence as set forth in SEQ ID NO:16.

According to one embodiment, the isolated polynucleotide comprises a nucleic acid sequence encoding a polypeptide comprising contiguous amino acids having at least 80% homology, preferably 90%, more preferably 95% and most preferably 100% homology to amino acids 151-168 of SEQ ID NO:16.

According to another aspect, the present invention provides an isolated polynucleotide encoding a novel splice variant of ANP having a nucleic acid sequence as set forth in SEQ ID NO:2.

According to yet another aspect, the present invention provides an isolated polynucleotide complementary to the nucleotide sequence set forth in SEQ ID NO:2.

In another embodiment, the present invention relates to bridges, tails, heads and/or insertions, and/or analogs, homologs and derivatives of the novel splice variants of the present invention. Such bridges, tails, heads and/or insertions are described in greater detail below with regard to the Examples.

As used herein a “tail” refers to a peptide sequence at the end of an amino acid sequence that is unique to a splice variant according to the present invention. Therefore, a splice variant having such a tail may optionally be considered as a chimera, in that at least a first portion of the splice variant is typically highly homologous (often 100% identical) to a portion of the corresponding known protein, while at least a second portion of the variant comprises the tail.

As used herein “an edge portion” refers to a connection between two portions of a splice variant according to the present invention that were not joined in the wild type or known protein. An edge may optionally arise due to a join between the above “known protein” portion of a variant and the tail, for example, and/or may occur if an internal portion of the wild type sequence is no longer present, such that two portions of the sequence are now contiguous in the splice variant that were not contiguous in the known protein. A “bridge” may optionally be an edge portion as described above, but may also include a join between a head and a “known protein” portion of a variant, or a join between a tail and a “known protein” portion of a variant, or a join between an insertion and a “known protein” portion of a variant.

According to additional aspects, the present invention provides vectors, cells, liposomes and compositions comprising the isolated nucleic acids of this invention.

According to additional aspects, the present invention provides pharmaceutical compositions comprising the novel splice variant polypeptides of this invention.

According to one embodiment, the present invention provides a pharmaceutical composition comprising as an active ingredient an ANP—splice variant polypeptide having an amino acid sequence as set forth in anyone of SEQ ID NOs:15, 16, 21-38.

According to one embodiment, the present invention provides a pharmaceutical composition comprising as an active ingredient an ANP—splice variant polynucleotide having a nucleic acid sequence as set forth in anyone of SEQ ID NOs:1 or 2.

According to another aspect, the present invention provides a method for treating an ANP-related disease, comprising administering to a subject in need thereof a therapeutically effective amount of a pharmaceutical composition comprising an active ingredient selected from the group consisting of a variant peptide or protein, a nucleic acid encoding the variant, an expression vector containing the nucleic acid sequence encoding the variant, and a host cell containing the expression vector.

According to certain preferred embodiments, the present invention provides a method for treating an ANP-related disease, comprising administering to a subject in need thereof a therapeutically effective amount of a pharmaceutical composition comprising as an active ingredient an ANP variant HUMCDDANF_{—}1_P5, having the amino acid sequence as set forth in SEQ ID NO:16 and active fragments and derivatives thereof.

According to other certain preferred embodiments the present invention provides a method for treating an ANP-related disease, comprising administering to a subject in need thereof a therapeutically effective amount of a pharmaceutical composition comprising as an active ingredient an ANP variant HUMCDDANF_{—}1_P4, having the amino acid sequence as set forth in SEQ ID NO:15 and active fragments and derivatives thereof.

According to yet other preferred embodiments the present invention provides a method for treating an ANP-related disease, comprising administering to a subject in need thereof a therapeutically effective amount of a pharmaceutical composition comprising as an active ingredient a cleavage product of an ANP variant selected from the group consisting of ANP-45 (SEQ ID NO:22); ANP-49 (SEQ ID NO:21): ANP-79 (SEQ ID NO:26) and ANP-83 (SEQ ID NO:25).

According to yet other preferred embodiments the present invention provides a method for treating an ANP-related disease, comprising administering to a subject in need thereof a therapeutically effective amount of a pharmaceutical composition comprising as an active ingredient a cleavage product of an ANP variant selected from the group consisting of SEQ ID NOs:21-36. According to the present invention, the ANP protein variants and polypeptides derived therefrom have agonistic mode of action. Thus, “an ANP-related disease” or a “disease wherein ANP is involved” refer to a disease in which ANP activity plays a favorable role, such that treating the disease may involve agonistic activity and/or raising ANP activity and/or expression.

In particular, diseases or conditions amenable to treatment with the splice variants of the invention include, but are not limited to:
cardiovascular diseases, including but not limited to acute or chronic heart failure, stroke, ischemic stroke (thrombotic, embolic, lacunar and hypoperfusion types of strokes), hemorrhagic stroke or transient ischemic attacks, sudden cardiac death from arrhythmia or any other heart related conditions, conditions that lead to heart failure including but not limited to myocardial infarction, angina pectoris (stable and unstable), arrhythmias, valvular diseases, conditions that cause atrial and or ventricular wall volume overload, systemic arterial hypertension, pulmonary hypertension, pulmonary embolism, respiratory distress syndrome—adult or other, conditions in which vasorelaxation or vasodilatation is efficacious, conditions in which diuresis is efficacious, asthma, obstructive lung disease, COPD (chronic obstructive pulmonary disease), cardiomyopathy, myocarditis, congestive heart failure, CVS diseases, atrial and/or ventricular septal defects. According to further aspects of the present invention, ANP variants of the present invention can be useful as natriuretics, diuretics, vasodilators and/or modulators of the renin-angiotensin-aldosterone system by reducing cardiac filling pressures and improving dyspnea.
renal function related disorders, such as diuresis and natriuresis for kidney related diseases, including but not limited to nephrotic syndrome, hepatic cirrhosis, pulmonary disease and acute or chronic renal failure, chronic kidney failure with residual kidney functions. According to further aspects of the present invention, ANP variants of the present invention can be useful as natriuretics, diuretics, vasodilators and/or modulators of the renin-angiotensin-aldosterone system; for treating cardiac insufficiency, more specifically with oedematosis and sodium retention, oliguric renal failure, blood pressure disregulation and ascites in chronic liver diseases, vasopressin disregulation, posterior pituitary malfunction, and the psychotropic effects induced thereby; and/or for treating chronic renal insufficiency by stimulating renal rest function, useful e.g. for increasing dialysis-free intervals and improving excretion of essential urine components;
cancer, such as brain cancer or adenocarcinoma, including but not limited to breast adenocarcinomas, colon adenocarcinomas, prostate adenocarcinomas and lung cancers such as small cell and squamous cell lung adenocarcinomas. Without wishing to be bound by a single theory, ANP variants of the present invention can decrease the number of adenocarcinoma cells and have antiproliferative functions for treatment of cancer.
fibrotic and inflammatory or allergic responses and diseases leading to hypertrophic and remodeling responses in heart and vasculature, including but not limited to myointimal proliferation in atherosclerosis, restenosis induced by angioplasty or vascular reconstructive surgery, glomerulonephritis, glomerulosclerosis or other diseases involving vascular cell proliferation. Without wishing to be bound by a single theory, ANP variants of the present invention might have a protective role for end-organ damage by counteracting fibrotic and inflammatory responses leading to hypertrophic and remodeling responses in heart and vasculature, or exhibiting bronchodilatory and anti-inflammatory activity
obesity. Without wishing to be bound by a single theory, ANP variants of the present invention might have a potent lipolytic effect.
bone elongation in situations of abnormal bone growth or short stature. According to further aspects of the present invention, ANP variants of the present invention can be used alone or in combination with growth protein for bone elongation in situations of abnormal bone growth or short stature.
keratoconjunctival failure, including but not limited to dry eye, corneal epithelial abrasion and corneal ulcer.
regulating proliferation and/or survival of neurons. According to certain embodiments of the present invention, ANP variants of the present invention can be used to treat neuronal death and/or injury, or cancer.

These and additional features of the invention will be better understood in conjunction with the figures description, examples and claims which follow.

### BRIEF DESCRIPTION OF DRAWINGS

FIG. 1 presents ANP peptides sequences. The unique amino acids in the novel ANP variants-derived peptides are marked in lower case and bold. The conserved Cysteines are marked in lower case and bold. The numbers in the parentheses refer to the positions of the amino acids of the peptide, numbered according to the ANP precursor (SwissProt Accession: ANF_HUMAN, SEQ ID NO:13). The additional number within the parentheses, if any, refers to the number of unique amino acids, marked in lower case and bold.

FIG. 2 shows the amino acid sequence of ANP79_Fc (SEQ ID NO:37) (FIG. 2A) and ANP 83_Fc (SEQ ID NO:38) (FIG. 2B). The signal peptide sequence is underlined; the ANP sequence-linker is shown in bold; the Fc portion is shown in Italic.

FIG. 3 shows a schematic diagram of the ANP variants. The upper sequence refers to the known wild type ANP pro-peptide (SEQ ID NO:13). ANP variant 2 (SEQ ID NO:15) and the derived ANP-79 variant (SEQ ID NO:26) are shown in the middle; ANP variant 1 (SEQ ID NO:16) and the derived ANP-45 variant (SEQ ID NO: 22) are at the bottom of the scheme. Exons are represented by white boxes while introns are represented by two headed arrows. Proteins are shown in boxes with upper right to lower left fill. The unique regions are represented by dashed-frame boxes. The peptide region is indicated.

FIG. 4 shows the performance of a classifier predicting protein cleavage sites trained on Swiss-Prot version 42 and tested on version 47.4, as described in Example 1.

FIG. 5 shows a histogram demonstrating differential expression of the HUMCDD_ANF cluster in brain malignant tumors versus normal tissue expression. “GEN” stands for general and “EPI” stands for epithelial cells, as described in the Example section hereinbelow.

FIG. 6 shows amino acid sequence alignment of ANP variant HUMCDDANF_{—}1_P4 (SEQ ID NO:15) to known ANP sequences. FIG. 6A shows Alignment of: HUMCDDANF_{—}1_P4 (SEQ ID NO:15) to ANF_HUMAN (SEQ ID NO:13). FIG. 6B shows Alignment of: HUMCDDANF_{—}1_P4 (SEQ ID NO:15) to NP_{—}006163 (SEQ ID NO:14).

FIG. 7 shows amino acid sequence alignment of ANP variant HUMCDDANF_{—}1_P5 (SEQ ID NO:16) to known ANP sequences. FIG. 7A shows Alignment of HUMCDDANF_{—}1_P5 (SEQ ID NO:16) to ANF_HUMAN (SEQ ID NO:13). FIG. 7B shows Alignment of HUMCDDANF_{—}1_P5 (SEQ ID NO:16) to NP_{—}006163 (SEQ ID NO:14).

FIG. 8 presents the gene structure of ANP as a bold black chain, with introns as plain bold line and exons as boxes. The intron between exon 2 and 3 (intron 2) is shown as plain black line, although it may be retained in the mature transcript. The various RNA transcripts are shown as plain-border boxes. The top RNA transcript depicts the known mRNA. The alternative variants 1 and 2 are shown below in that order as indicated. The corresponding pre-pro-peptides are depicted as boxes with upper left to lower right fill, below each transcript. The numbers indicate length of the proteins in amino-acids. There are two known human pre-pro-proteins created by a single nucleotide polymorphism (SNP), that differ by the absence or presence of a C-terminal Arginine-Arginine dipeptide, thus their lengths are 151 and 153 amino acids, respectively, as indicated for the known pre-pro-peptide. The unique amino acids of each pre-pro-protein ANP variant are depicted by a dashed frame and the length of the unique region is shown at its right side.

FIG. 9 demonstrates the RT-PCR validation of ANP Splice Variant 2 (SEQ ID NO:15). FIG. 9a presents the products of RT-PCR performed on mix of total RNA extracted as follows: line #1 from heart, bone, fibroblasts and brain tissues; line #2 from brain, kidney, heart, liver and testis tissues and line #3 from kidney, liver, ovary and blood. FIG. 9b presents the product of RT-PCR performed on total RNA extracted from a fibrotic heart. The PCR bands that represent the ANP splice variant 2 (SEQ ID NO:15) are indicated by black arrows. The upper band in FIG. 9b is probably a product of a genomic contamination. MW is the molecular weight marker.

FIG. 10 shows sequencing results of the ANP-variant 1 PCR product from failing heart, demonstrating exon 2 and exon 3a junction.

FIG. 11 presents a cleavage map of ANP pre-pro-peptide variants. The pre-pro-peptides are depicted as boxes. Known regions are depicted by boxes between 1 to 150 (the numbers indicating the amino acid position (according to ANP precursor sequence ANF_HUMAN, SEQ ID NO:13). The unique region in each variant is depicted by a box with its length shown at the right side. Semi-transparent areas represent parts that are cleaved out. Dotted lines indicate cleavage sites, the naming of which is noted by arrows and short descriptions (see also legend in insert). The amino acid at the C-terminal side of each cleavage site is denoted by its number (according to ANP precursor sequence ANF_HUMAN, SEQ ID NO:13). Amino acid 127 (cleavage site (C)) is denoted only once, for clarity. ° C.'s represent conserved cysteines that form a disulfide bridge. One to three amino acids that reside immediately C-terminal to amino acid 150 (according to ANP precursor sequence ANF_HUMAN, SEQ ID NO:13) are denoted in one-letter code.

FIG. 12 shows the results of cGMP accumulation after activation of membrane-bound guanylyl cyclase in isolated rat lung membranes, demonstrating that ANP-28 (SEQ ID NO:18) synthesized in-house has similar activity to human ANP from a commercial source, and that ANP-32 (urodilatin) (SEQ ID NO:17) synthesized in-house also activates cGMP accumulation. Furthermore the effect of the human peptides is compared to the effect of rat ANF on rat receptors.

FIG. 13 shows the results of cGMP accumulation in isolated rat lung membranes after activation of membrane-bound guanylyl cyclase, demonstrating the agonist-like activity of ANP variant peptides #3 (CGD-3, PT# 1072384, ANP45, SEQ ID NO:22), #4 (CGD-4, PT# 1072385, ANP49, SEQ ID NO:21) compared to the wild type peptides #1 (CGD-1, PT# 1072382, ANP28, SEQ ID NO:18), #2 (CGD-2, PT# 1072383, ANP32, SEQ ID NO:17) and rat ANF.

FIG. 14 shows the results of mean arterial pressure (MAP) at different time points as described in Example 7 hereinbelow. All values were normalized to the MAP value at the time of initiation of peptide infusion (30 min). The values represent the mean of the normalized values of 5 animals for each group at 0, 30, 60, 90 and 120 minutes.

FIG. 15 shows the absolute changes of mean arterial pressure, as described in Example 7 hereinbelow. The absolute changes were calculated as delta between MAP-infusion at the 60 min time point and MAP-baseline (at the 30 min time point). The values represent the mean±SEM of 5 animals for each group. *p<0.05 vs vehicle control.

FIG. 16 shows the results of the heart rate at different time points, as described in Example 7 hereinbelow. All values are expressed as delta between the measurement at the relevant time point and the baseline heart rate (30 min). The values represent the mean of 5 animals for each group at 0, 30, 60, 90 and 120 minutes. *p<0.05 vs vehicle control.

FIG. 17 shows the absolute changes of the heart rate, as described in Example 7 hereinbelow. The absolute changes were calculated as delta between the heart rate after infusion and the baseline heart rate. The values represent the mean±SEM of 5 animals for each group. All values (except vehicle control group) do not have significant variance vs. vehicle control.

FIG. 18 shows the results of the urine volume analysis, as described in Example 7 hereinbelow. The values represent the mean±SEM fold increase of 5 animals for each group at time intervals of 30-60 min, 60-90 min and 90-120 min. The reduction and variability in urine volume at the time intervals of 60-90 min and 90-120 min may be due to technical problems such as dehydration of the animals.

FIG. 19 shows the absolute changes of the urine volume, as described in Example 7 hereinbelow. The absolute changes were calculated as delta between the urine volume after infusion and the baseline urine volume. The values represent the mean±SEM of 5 animals for each group.

FIG. 20 shows the results of the urine sodium excretion analysis, as described in Example 7 hereinbelow. The values represent the mean±SEM of 5 animals for each group at time intervals of 0-30 min, 30-60 min, 60-90 min and 90-120 min. *p<0.05 vs vehicle control.

FIG. 21 shows the absolute changes in the urine sodium excretion, as described in Example 7 hereinbelow. The absolute changes were calculated as delta between the urine sodium excretion after infusion and the baseline urine sodium excretion. The values represent the mean±SEM of 5 animals for each group. *p<0.05 vs vehicle control.

### DESCRIPTION OF PREFERRED EMBODIMENTS

The present invention provides ANP variant proteins and polypeptides, which may be used for therapeutic applications.

Preferably, but not limiting, these protein and polypeptide variants and isolated polynucleotides encoding same are useful as therapeutic agents for ANP-related diseases. Diseases treatable by the ANP variants are described herein and are collectively referred to as “variant-treatable diseases”. According to the present invention, the ANP therapeutic variant proteins and the polypeptides derived therefrom has agonistic mode of action. A “variant-treatable” disease includes but is not limited to:
cardiovascular diseases, including but not limited to acute or chronic heart failure, stroke, ischemic stroke (thrombotic, embolic, lacunar and hypoperfusion types of strokes), hemorrhagic stroke or transient ischemic attacks, sudden cardiac death from arrhythmia or any other heart related conditions, conditions that lead to heart failure including but not limited to myocardial infarction, angina pectoris (stable and unstable), arrhythmias, valvular diseases, conditions that cause atrial and or ventricular wall volume overload, systemic arterial hypertension, pulmonary hypertension, pulmonary embolism, respiratory distress syndrome—adult or other, conditions in which vasorelaxation or vasodilatation is efficacious, conditions in which diuresis is efficacious, asthma, obstructive lung disease, COPD (chronic obstructive pulmonary disease), cardiomyopathy, myocarditis, congestive heart failure, CVS diseases, atrial and/or ventricular septal defects;
renal function related disorders, such as diuresis and natriuresis for kidney related diseases, including but not limited to nephrotic syndrome, hepatic cirrhosis, pulmonary disease and acute or chronic renal failure, chronic kidney failure with residual kidney functions, cardiac insufficiency, more specifically with oedematosis and sodium retention, oliguric renal failure, blood pressure disregulation and ascites in chronic liver diseases, vasopressin disregulation, posterior pituitary malfunction, and the psychotropic effects induced thereby; and/or chronic renal insufficiency;
cancer, such as brain cancer or adenocarcinoma, including but not limited to breast adenocarcinomas, colon adenocarcinomas, prostate adenocarcinomas and lung cancers such as small cell and squamous cell lung adenocarcinomas;
fibrotic and inflammatory or allergic responses and diseases leading to hypertrophic and remodeling responses in heart and vasculature, including but not limited to myointimal proliferation in atherosclerosis, restenosis induced by angioplasty or vascular reconstructive surgery, glomerulonephritis, glomerulosclerosis or other diseases involving vascular cell proliferation;
obesity;
bone elongation in situations of abnormal bone growth or short stature;
keratoconjunctival failure, including but not limited to dry eye, corneal epithelial abrasion and corneal ulcer;
regulating proliferation and/or survival of neurons.

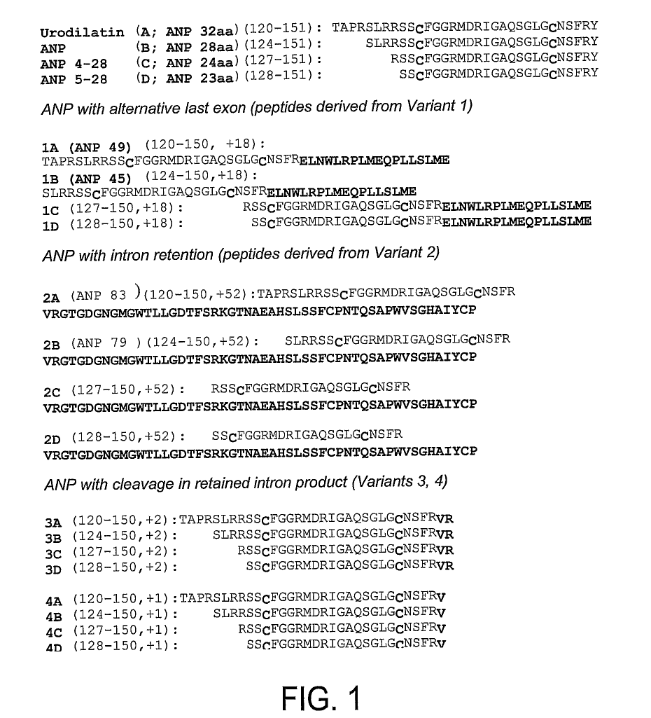

According to the present invention a novel transcript of ANP splice variant was identified: HUMCDDANF_{—}1_T4 (SEQ ID NO:2). This transcript encodes for a protein which is a variant of atrial natriuretic factor precursor protein: HUMCDDANF_{—}1_P5 (Variant 1, SEQ ID NO: 16). According to the present invention the HUMCDDANF_{—}1_P4 (Variant 2, SEQ ID NO:15, previously described by inventors of the present invention in International Patent Application PCT/IL2006/000676) undergoes further protein cleavage to produce corresponding peptides 2A, 2B, 2C, 2D, 3A, 3B, 3C, 3D, 4A, 4B, 4C, and 4D (SEQ ID NOs: 25-36, respectively). According to the present invention the HUMCDDANF_{—}1_P5 (Variant 1, SEQ ID NO:16) undergoes further protein cleavage to produce corresponding peptides 1A, 1B, 1C, 1D (SEQ ID NOs: 21-24, respectively). The sequences of the variant ANP peptides of the present invention are given in FIG. 1.

In FIG. 1 the unique amino acids in the novel ANP variants are marked in lower case and bold. The conserved Cysteins are marked in lower case and bold. The numbers in the parentheses refer to the positions of the amino acids of the peptide, numbered according to the ANP precursor (SwissProt Accession: ANF_HUMAN, SEQ ID NO:13). The additional number within the parentheses, if any, refers to the number of unique amino acids, marked in lower case and bold.

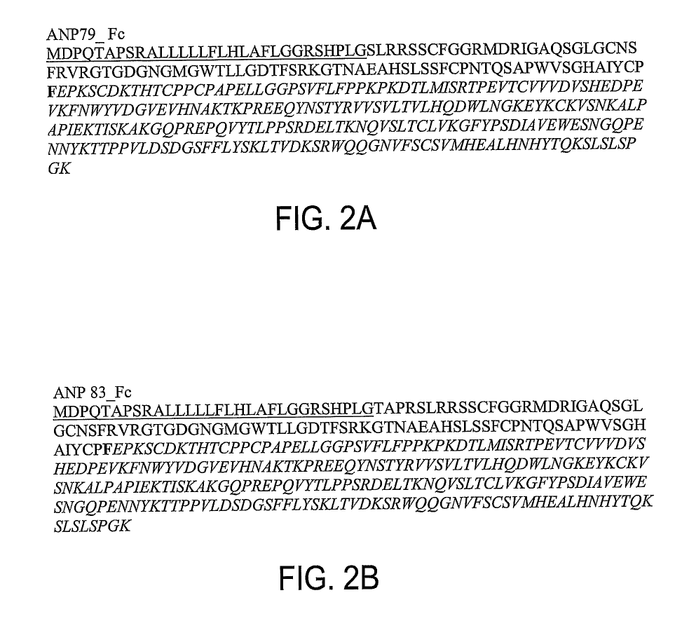

The ANP variants may optionally and preferably be used in Fc form, according to the following structure: signal peptide-ANP sequence-linker-Tag, wherein the Tag is the Fc portion (in this example, it is preferably glycosylated). ANP79_Fc (SEQ ID NO:37) sequence is shown in FIG. 2A and ANP 83_Fc (SEQ ID NO:38) sequence is shown in FIG. 2B. The signal peptide sequence is underlined; the ANP sequence-linker is shown in bold; the Fc portion is shown in Italic.

Table 1 presents proteins and peptides and their names (WT indicates that peptide was derived from the known or wild type ANP sequence). The described ANP variants are significantly longer than hitherto known wild type ANP proteins. This may result in more stable peptides with a longer half-life. The unique C terminal tail may confer improved receptor specificity and improved pharmacokinetic and pharmacodynamic properties.

TABLE 1

| | | SEQ ID |
|---|---|---|
| Name | Peptide sequence | NO: |
| ANP 28 (WT) | SLRRSSCFGGRMDRIGAQSGLGCNSFRY | 18 |
| ANP 32 (WT | TAPRSLRRSSCFGGRMDRIGAQSGLGCNSFRY | 17 |
| urodilatin) | | |
| ANP 45 | SLRRSSCFGGRMDRIGAQSGLGCNSFRELNWL | 22 |
| (variant) | RPLMEQPLLSLME | |
| ANP 49) | TAPRSLRRSSCFGGRMDRIGAQSGLGCNSFRE | 21 |
| (variant) | LNWLRPLMEQPLLSLME | |
| ANP 79 | SLRRSSCFGGRMDRIGAQSGLGCNSFRVRGTG | 26 |
| (variant) | DGNGMGWTLLGDTFSRKGTNAEAHSLSSFCPN | |
| | TQSAPWVSGHAIYCP | |
| ANP 83 | TAPRSLRRSSCFGGRMDRIGAQSGLGCNSFRV | 25 |
| (variant) | RGTGDGNGMGWTLLGDTFSRKGTNAEAHSLSS | |
| | FCPNTQSAPWVSGHAIYCP | |

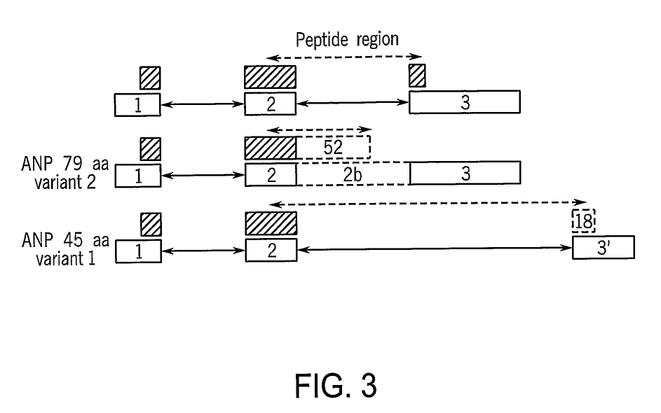

The structure of the above variants is schematically described by the diagram of FIG. 3. The upper sequence refers to the known wild type ANP pro-peptide (SEQ ID NO: 13). The ANP variant 2 (SEQ ID NO: 15) and the derived ANP-79 (SEQ ID NO: 26) are shown in the middle; the ANP variant 1 (SEQ ID NO:16) and the derived ANP-45 (SEQ ID NO: 22) are at the bottom of the scheme. Exons are represented by white boxes while introns are represented by two headed arrows. Proteins are shown in boxes with upper right to lower left fill. The unique regions are represented by dashed-frame boxes. The peptide region is indicated.

According to certain embodiments, the present invention provides cleavage products of the chimeric polypeptide designated HUMCDDANF_{—}1_P4 (SEQ ID NO:15), described in PCT/IL2006/000676. HUMCDDANF_{—}1_P4 (SEQ ID NO:15) is a chimeric protein comprising a first amino acid sequence being at least 90% homologous to the amino acid sequence MSSFSTTTVSFLLLLAFQLLGQTRANPMYNAVSNADLMDFKNLLDHLEEKMPLE DEVVPPQVLSEPNEEAGAALSPLPEVPPWTGEVSPAQRDGGALGRGPWDSSDRS ALLKSKLRALLTAPRSLRRSSCFGGRMDRIGAQSGLGCNSFR corresponding to amino acids 1-150 of ANF_HUMAN (SEQ ID NO:13), which also corresponds to amino acids 1-150 of HUMCDDANF_{—}1_P4 (SEQ ID NO:15), and a second amino acid sequence being at least 70%, optionally at least 80%, preferably at least 85%, more preferably at least 90% and most preferably at least 95% homologous to a polypeptide having the sequence VRGTGDGNGMGWTLLGDTFSRKGTNAEAHSLS SFCPNTQSAPWVSGHAIYCP (SEQ ID NO:39) corresponding to amino acids 151-202 of HUMCDDANF_{—}1_P4 (SEQ ID NO:15), wherein said first amino acid sequence and second amino acid sequence are contiguous and in a sequential order.

An isolated polypeptide designated an edge portion of HUMCDDANF_{—}1_P4 (SEQ ID NO:15), comprises an amino acid sequence being at least 70%, optionally at least about 80%, preferably at least about 85%, more preferably at least about 90% and most preferably at least about 95% homologous to the sequence VRGTGDGNGMGWTLLGDTFSRKGTNAEAHSLSSFCPNTQSAPWVSGHAIYCP (SEQ ID NO:39) of HUMCDDANF_{—}1_P4.

A bridge portion of HUMCDDANF_{—}1_P4 (SEQ ID NO:15), comprises a polypeptide having a length “n”, wherein n is at least about 10 amino acids in length, optionally at least about 15 amino acids in length, preferably at least about 20 amino acids in length, more preferably at least about 25 amino acids in length and most preferably at least about 30 amino acids in length, wherein at least two amino acids comprise RV, having a structure as follows (numbering according to HUMCDDANF_{—}1_P4): a sequence starting from any of amino acid numbers 150-x to 150; and ending at any of amino acid numbers 151+((n−2)−x), in which x varies from 0 to n−2.

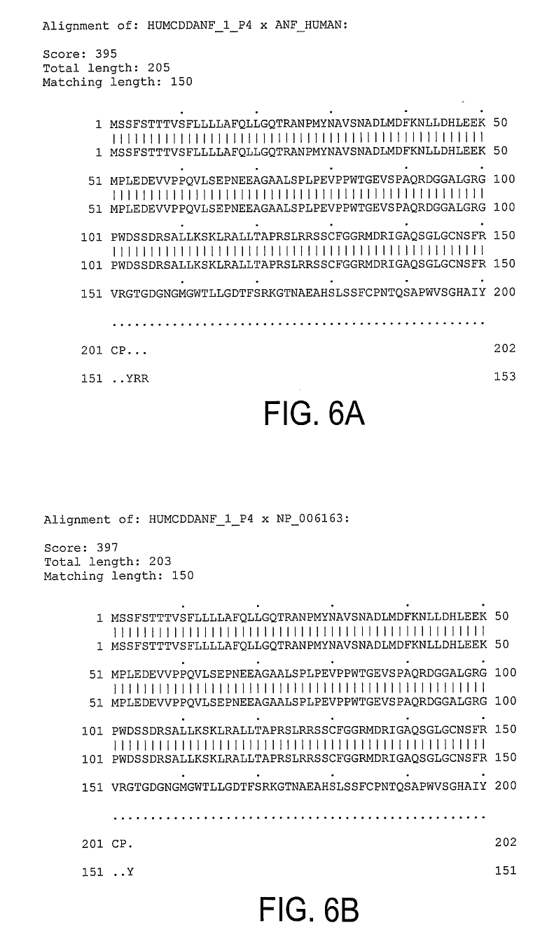

FIG. 6A shows an alignment between the known atrial natriuretic factor precursor protein ANF_HUMAN (SEQ ID NO:13) and variant HUMCDDANF_{—}1_P4 (SEQ ID NO:15).

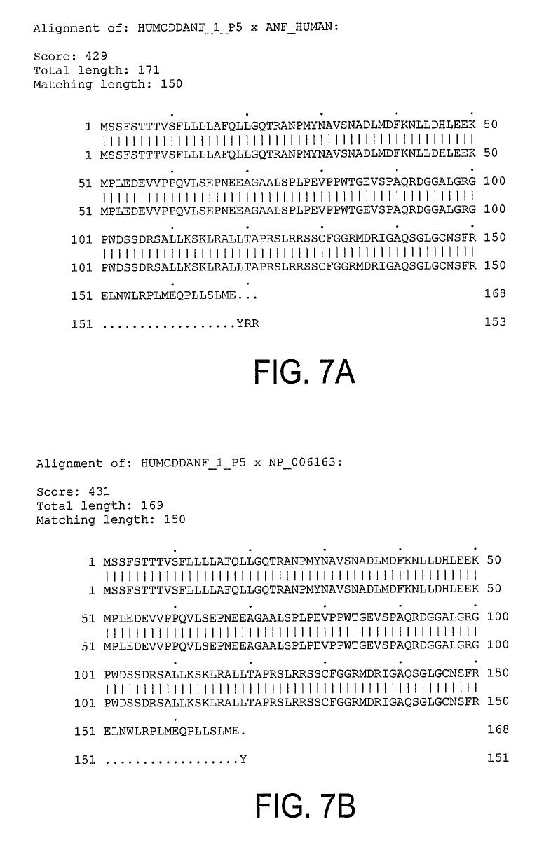

Variant protein HUMCDDANF_{—}1_P5 (SEQ ID NO:16) according to the present invention has an amino acid sequence as set forth in SEQ ID NO:16; it is encoded by transcript HUMCDDANF_{—}1_T4 (SEQ ID NO:2). An alignment between the known atrial natriuretic factor precursor protein ANF_HUMAN (SEQ ID NO:13) and the variant protein HUMCDDANF_{—}1_P5 (SEQ ID NO:16) of the present invention is shown in FIG. 7A.

According to one embodiment, the present invention provides an isolated chimeric polypeptide designated HUMCDDANF_{—}1_P5 (SEQ ID NO:16), comprising a first amino acid sequence being at least 90% homologous to MSSFSTTTVSFLLLLAFQLLGQTRANPMYNAVSNADLMDFKNLLDHLEEKMPLE DEVVPPQVLSEPNEEAGAALSPLPEVPPWTGEVSPAQRDGGALGRGPWDSSDRS ALLKSKLRALLTAPRSLRRSSCFGGRMDRIGAQSGLGCNSFR corresponding to amino acids 1-150 of ANF_HUMAN (SEQ ID NO:13), which also corresponds to amino acids 1-150 of HUMCDDANF_{—}1_P5 (SEQ ID NO:16), and a second amino acid sequence being at least 70%, optionally at least 80%, preferably at least 85%, more preferably at least 90% and most preferably at least 95% homologous to a polypeptide having the sequence ELNWLRPLMEQPLLSLME (SEQ ID NO:40) corresponding to amino acids 151-168 of HUMCDDANF_{—}1_P5 (SEQ ID NO:16), wherein said first amino acid sequence and second amino acid sequence are contiguous and in a sequential order.

An isolated polypeptide designated an edge portion of HUMCDDANF_{—}1_P5 (SEQ ID NO:16), comprising an amino acid sequence being at least 70%, optionally at least about 80%, preferably at least about 85%, more preferably at least about 90% and most preferably at least about 95% homologous to the sequence ELNWLRPLMEQPLLSLME (SEQ ID NO:40) of HUMCDDANF_{—}1_P5 (SEQ ID NO:16).

A bridge portion of HUMCDDANF_{—}1_P5 (SEQ ID NO:16), comprising a polypeptide having a length “n”, wherein n is at least about 10 amino acids in length, optionally at least about 15 amino acids in length, preferably at least about 20 amino acids in length, more preferably at least about 25 amino acids in length and most preferably at least about 30 amino acids in length, wherein at least two amino acids comprise RE, having a structure as follows (numbering according to HUMCDDANF_{—}1_P5 (SEQ ID NO:16)): a sequence starting from any of amino acid numbers 150-x to 150; and ending at any of amino acid numbers 151+((n−2)−x), in which x varies from 0 to n−2.

According to the present invention, the ANP protein variants and polypeptides derived therefrom according to the teaching of the present invention have agonistic mode of action. Thus, “diseases wherein ANP is involved” refers to a disease in which ANP activity plays a favorable role, such that treating the disease may involve agonist and/or raising ANP activity and/or expression.

### Description of the Methodology Undertaken to Uncover the Biomolecular Sequences of the Present Invention

Human ESTs and cDNAs were obtained from GenBank versions 145 (Dec. 23, 2004 ftp://ftp.ncbi.nih.gov/genbank/release.notes/gbl45136.release.notes) and NCBI genome assembly of Aug. 26, 2005 (Build 35). Novel splice variants were predicted using the LEADS clustering and assembly system as described in U.S. Pat. No. 6,625,545 and U.S. patent application Ser. No. 10/426,002, publised as US20040101876, both of which are hereby incorporated by reference as if fully set forth herein. Briefly, the software cleans the expressed sequences from repeats, vectors and immunoglobulins. It then aligns the expressed sequences to the genome taking alternative splicing into account and clusters overlapping expressed sequences into “clusters” that represent genes or partial genes.

These were annotated using the GeneCarta (Compugen, Tel-Aviv, Israel) platform. The GeneCarta platform includes a rich pool of annotations, sequence information (particularly of spliced sequences), chromosomal information, alignments, and additional information such as SNPs, gene ontology terms, expression profiles, functional analyses, detailed domain structures, known and predicted proteins and detailed homology reports.

Brief description of the methodology used to obtain annotative sequence information is summarized infra (for detailed description see U.S. patent application Ser. No. 10/426,002, published as US20040101876).

The ontological annotation approach—An ontology refers to the body of knowledge in a specific knowledge domain or discipline such as molecular biology, microbiology, immunology, virology, plant sciences, pharmaceutical chemistry, medicine, neurology, endocrinology, genetics, ecology, genomics, proteomics, cheminformatics, pharmacogenomics, bioinformatics, computer sciences, statistics, mathematics, chemistry, physics and artificial intelligence.

An ontology includes domain-specific concepts—referred to, herein, as sub-ontologies. A sub-ontology may be classified into smaller and narrower categories. The ontological annotation approach is effected as follows.

First, biomolecular (i.e., polynucleotide or polypeptide) sequences are computationally clustered according to a progressive homology range, thereby generating a plurality of clusters each being of a predetermined homology of the homology range.

Progressive homology is used to identify meaningful homologies among biomolecular sequences and to thereby assign new ontological annotations to sequences, which share requisite levels of homologies. Essentially, a biomolecular sequence is assigned to a specific cluster if displays a predetermined homology to at least one member of the cluster (i.e., single linkage). A “progressive homology range” refers to a range of homology thresholds, which progress via predetermined increments from a low homology level (e.g. 35%) to a high homology level (e.g. 99%).

Following generation of clusters, one or more ontologies are assigned to each cluster. Ontologies are derived from an annotation preassociated with at least one biomolecular sequence of each cluster; and/or generated by analyzing (e.g., text-mining) at least one biomolecular sequence of each cluster thereby annotating biomolecular sequences.

The hierarchical annotation approach—“Hierarchical annotation” refers to any ontology and subontology, which can be hierarchically ordered, such as, a tissue expression hierarchy, a developmental expression hierarchy, a pathological expression hierarchy, a cellular expression hierarchy, an intracellular expression hierarchy, a taxonomical hierarchy, a functional hierarchy and so forth.

The hierarchical annotation approach is effected as follows. First, a dendrogram representing the hierarchy of interest is computationally constructed. A “dendrogram” refers to a branching diagram containing multiple nodes and representing a hierarchy of categories based on degree of similarity or number of shared characteristics.

Each of the multiple nodes of the dendrogram is annotated by at least one keyword describing the node, and enabling literature and database text mining, such as by using publicly available text mining software. A list of keywords can be obtained from the GO Consortium (www.geneontlogy.org). However, measures are taken to include as many keywords, and to include keywords which might be out of date. For example, for tissue annotation, a hierarchy is built using all available tissue/libraries sources available in the GenBank, while considering the following parameters: ignoring GenBank synonyms, building anatomical hierarchies, enabling flexible distinction between tissue types (normal versus pathology) and tissue classification levels (organs, systems, cell types, etc.).

In a second step, each of the biomolecular sequences is assigned to at least one specific node of the dendrogram.

The biomolecular sequences can be annotated biomolecular sequences, unannotated biomolecular sequences or partially annotated biomolecular sequences.

Annotated biomolecular sequences can be retrieved from pre-existing annotated databases as described hereinabove.

For example, in GenBank, relevant annotational information is provided in the definition and keyword fields. In this case, classification of the annotated biomolecular sequences to the dendrogram nodes is directly effected. A search for suitable annotated biomolecular sequences is performed using a set of keywords which are designed to classify the biomolecular sequences to the hierarchy (i.e., same keywords that populate the dendrogram).

In cases where the biomolecular sequences are unannotated or partially annotated, extraction of additional annotational information is effected prior to classification to dendrogram nodes. This can be effected by sequence alignment, as described hereinabove. Alternatively, annotational information can be predicted from structural studies. Where needed, nucleic acid sequences can be transformed to amino acid sequences to thereby enable more accurate annotational prediction.

Finally, each of the assigned biomolecular sequences is recursively classified to nodes hierarchically higher than the specific nodes, such that the root node of the dendrogram encompasses the full biomolecular sequence set, which can be classified according to a certain hierarchy, while the offspring of any node represent a partitioning of the parent set.

For example, a biomolecular sequence found to be specifically expressed in “rhabdomyosarcoma”, will be classified also to a higher hierarchy level, which is “sarcoma”, and then to “Mesenchymal cell tumors” and finally to a highest hierarchy level “Tumor”. In another example, a sequence found to be differentially expressed in endometrium cells, will be classified also to a higher hierarchy level, which is “uterus”, and then to “women genital system” and to “genital system” and finally to a highest hierarchy level “genitourinary system”. The retrieval can be performed according to each one of the requested levels.

Annotating gene expression according to relative abundance—Spatial and temporal gene annotations are also assigned by comparing relative abundance in libraries of different origins. This approach can be used to find genes, which are differentially expressed in tissues, pathologies and different developmental stages. In principal, the presentation of a contigue in at least two tissues of interest is determined and significant over or under representation of the contigue in one of the at least two tissues is assessed to identify differential expression. Significant over or under representation is analyzed by statistical pairing.

Annotating spatial and temporal expression can also be effected on splice variants. This is effected as follows. First, a contigue which includes exonal sequence presentation of the at least two splice variants of the gene of interest is obtained. This contigue is assembled from a plurality of expressed sequences. Then, at least one contigue sequence region, unique to a portion (i.e., at least one and not all) of the at least two splice variants of the gene of interest, is identified. Identification of such unique sequence region is effected using computer alignment software. Finally, the number of the plurality of expressed sequences in the tissue having the at least one contigue sequence region is compared with the number of the plurality of expressed sequences not-having the at least one contigue sequence region, to thereby compare the expression level of the at least two splice variants of the gene of interest in the tissue.

Data concerning therapies, indications and possible pharmacological activities of the polypeptides of the present invention was obtained from PharmaProject (PJB Publications Ltd 2003 http://www.pjbpubs.com/cms.asp?pageid=340) and public databases, including LocusLink (http://www.genelynx.org/cgi-bin/resource?res=locuslink) and Swissprot (http://www.ebi.ac.uk/swissprot/index.html). Functional structural analysis of the polypeptides of the present invention was effected using Interpro domain analysis software (Interpro default parameters, the analyses that were run are HMMPfam, HMMSmart, ProfileScan, FprintScan, and BlastProdom). Subcellular localization was analyzed using ProLoc software (Einat Hazkani-Covo, Erez Y. Levanon, Galit Rotman, Dan Graur, Amit Novik. Evolution of multicellularity in metazoa: comparative analysis of the subcellular localization of proteins in Saccharomyces, Drosophila and Caenorhabditis. Cell Biology International (2004; 28(3): 171-8).

### Identifying Gene Products by Interspecies Sequence Comparison

The present inventors have designed and configured a method of predicting gene expression products based on interspecies sequence comparison. Specifically, the method is based on the identification of conserved alternatively spliced exons for which there might be no supportive expression data.

Alternatively spliced exons have unique characteristics differentiating them from constitutively spliced ones. Using machine-learning techniques a combination of such characteristics was elucidated that defines alternatively spliced exons with very high probability. Any human exon having this combination of characteristics is therefore predicted to be alternatively spliced. Using this method, the present inventors were able to detect putative splice variants that are not supported by human ESTs.

The method is effected as follows. First, alternatively spliced exons of a gene of interest are identified by scoring exon sequences of the gene of interest according to at least one sequence parameter as follows: (i) exon length—conserved alternatively spliced exons are relatively shorter than constitutively spliced ones; (ii) division by 3—alternatively spliced exons are cassette exons that are sometimes inserted and sometimes skipped; Since alternatively spliced exons frequently contain sequences that regulate their splicing important parameters for scoring alternatively spliced exons include (iii) conservation level to a non-human ortholohgous sequence; (iv) length of conserved intron sequences upstream of each of the exon sequences; (v) length of conserved intron sequences downstream of each of the exon sequences; (vi) conservation level of the intron sequences upstream of each of the exon sequences; and (vii) conservation level of the intron sequences downstream of each of the exon sequences.

Exon sequences scoring above a predetermined threshold represent alternatively spliced exons of the gene of interest.

Once alternatively spliced exons are identified, the chromosomal location of each of the alternatively spliced exons is analyzed with respect to coding sequence of the gene of interest to thereby predict expression products of the gene of interest. When performed along with computerized means, mass prediction of gene products can be effected.

In addition, for identifying new gene products by interspecies sequence comparison, the expressed sequences derived from non-human species can be used for new human splice variants prediction.

### Prediction of Cellular Localization

Information given in the text with regard to cellular localization was determined according to four different software programs: (i) tmhmm (from Center for Biological Sequence Analysis, Technical University of Denmark DTU, http://www.cbs.dtu.dk/services/TMHMM/TMHMM2.0b.guide.php) or (ii) tmpred (from EMBnet, maintained by the ISREC Bionformatics group and the LICR Information Technology Office, Ludwig Institute for Cancer Research, Swiss Institute of Bioinformatics, http://www.ch.embnet.org/software/TMPRED_form.html) for transmembrane region prediction; (iii) signalp_hmm and (iv) signalp_nn (both from Center for Biological Sequence Analysis, Technical University of Denmark DTU, http://www.cbs.dtu.dk/services/SignalP/background/prediction.php) for signal peptide prediction. The terms “signalp_hmm” and “signalp_nn” refer to two modes of operation for the program SignalP: hmm refers to Hidden Markov Model, while nn refers to neural networks. Localization was also determined through manual inspection of known protein localization and/or gene structure, and the use of heuristics by the individual inventor. In some cases for the manual inspection of cellular localization prediction inventors used the ProLoc computational platform (Einat Hazkani-Covo, Erez Levanon, Galit Rotman, Dan Graur and Amit Novik; (2004) Evolution of multicellularity in metazoa: comparative analysis of the subcellular localization of proteins in Saccharomyces, Drosophila and Caenorhabditis. Cell Biology International 2004; 28(3):171-8), which predicts protein localization based on various parameters including, protein domains (e.g., prediction of trans-membranous regions and localization thereof within the protein), pI, protein length, amino acid composition, homology to pre-annotated proteins, recognition of sequence patterns which direct the protein to a certain organelle (such as, nuclear localization signal, NLS, mitochondria localization signal), signal peptide and anchor modeling and using unique domains from Pfam that are specific to a single compartment.

### Single Nucleotide Polymorphisms

Information is given in the text with regard to SNPs (single nucleotide polymorphisms). A description of the abbreviations is as follows. “T->C”, for example, means that the SNP results in a change at the position given in the table from T to C. Similarly, “M->Q”, for example, means that the SNP has caused a change in the corresponding amino acid sequence, from methionine (M) to glutamine (Q). If, in place of a letter at the right hand side for the nucleotide sequence SNP, there is a space, it indicates that a frameshift has occurred. A frameshift may also be indicated with a hyphen (-). A stop codon is indicated with an asterisk at the right hand side (*). As part of the description of an SNP, a comment may be found in parentheses after the above description of the SNP itself. This comment may include an FTId, which is an identifier to a SwissProt entry that was created with the indicated SNP. An FTId is a unique and stable feature identifier, which allows construction of links directly from position-specific annotation in the feature table to specialized protein-related databases. The FTId is always the last component of a feature in the description field, as follows: FTId=XXX_number, in which XXX is the 3-letter code for the specific feature key, separated by an underscore from a 6-digit number. In the table of the amino acid mutations of the wild type proteins of the selected splice variants of the invention, the header of the first column is “SNP position(s) on amino acid sequence”, representing a position of a known mutation on amino acid sequence. For each given SNP, it was determined whether it was previously known by using dbSNP build 122 from NCBI, released on Aug. 13, 2004.

Information given in the text with regard to the Homology to the wild type was determined by Smith-Waterman version 5.1.2 Using Special (non default) parameters as follows:

-model=sw.model

### -GAPEXT=0

### -GAPOP=100.0

-MATRIX=blosum100

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). All of these are hereby incorporated by reference as if fully set forth herein. As used herein, the following terms have the meanings ascribed to them unless specified otherwise.

### Assays, Terms and Definitions

The term “biologically active”, as used herein, refers to a protein having structural, regulatory, or biochemical functions of a naturally occurring molecule. Likewise, “immunologically active” refers to the capability of the natural, recombinant, or synthetic ligand, or any oligopeptide thereof, to induce a specific immune response in appropriate animals or cells and to bind with specific antibodies.

The term “modulate”, as used herein, refers to a change in the activity of at least one receptor mediated activity. For example, modulation may cause an increase or a decrease in protein activity, binding characteristics, or any other biological, functional or immunological properties of a ligand.

As used herein the phrase “disease” includes any type of pathology and/or damage, including both chronic and acute damage, as well as a progress from acute to chronic damage.

### Nucleic Acids

A “nucleic acid fragment” or an “oligonucleotide” or a “polynucleotide” are used herein interchangeably to refer to a polymer of nucleic acid residues. A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is composed of genomic and cDNA sequences. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Thus, the present invention encompasses nucleic acid sequences described hereinabove; fragments thereof, sequences hybridizable therewith, sequences homologous thereto (e.g., at least 50%, at least 55%, at least 60%, at least 65%, at least 70%, at least 75%, at least 80%, at least 85%, at least 95% or more say 100% identical to the nucleic acid sequences set forth below), sequences encoding similar polypeptides with different codon usage, altered sequences characterized by mutations, such as deletion, insertion or substitution of one or more nucleotides, either naturally occurring or man induced, either randomly or in a targeted fashion. The present invention also encompasses homologous nucleic acid sequences (i.e., which form a part of a polynucleotide sequence of the present invention) which include sequence regions unique to the polynucleotides of the present invention.

In cases where the polynucleotide sequences of the present invention encode previously unidentified polypeptides, the present invention also encompasses novel polypeptides or portions thereof, which are encoded by the isolated polynucleotide and respective nucleic acid fragments thereof described hereinabove.

Thus, the present invention also encompasses polypeptides encoded by the polynucleotide sequences of the present invention. The present invention also encompasses homologues of these polypeptides, such homologues can be at least 50%, at least 55%, at least 60%, at least 65%, at least 70%, at least 75%, at least 80%, at least 85%, at least 95% or more say 100% homologous to the amino acid sequences set forth below, as can be determined using BlastP software of the National Center of Biotechnology Information (NCBI) using default parameters. Finally, the present invention also encompasses fragments of the above described polypeptides and polypeptides having mutations, such as deletions, insertions or substitutions of one or more amino acids, either naturally occurring or man induced, either randomly or in a targeted fashion.

As mentioned hereinabove, biomolecular sequences uncovered using the methodology of the present invention can be efficiently utilized as tissue or pathological markers and as putative drugs or drug targets for treating or preventing a disease.

Oligonucleotides designed for carrying out the methods of the present invention for any of the sequences provided herein (designed as described above) can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art.

Oligonucleotides used according to this aspect of the present invention are those having a length selected from a range of about 10 to about 200 bases preferably about 15 to about 150 bases, more preferably about 20 to about 100 bases, most preferably about 20 to about 50 bases.

The oligonucleotides of the present invention may comprise heterocylic nucleosides consisting of purines and the pyrimidines bases, bonded in a 3′ to 5′ phosphodiester linkage.

Preferably used oligonucleotides are those modified in either backbone, internucleoside linkages or bases, as is broadly described hereinunder. Such modifications can oftentimes facilitate oligonucleotide uptake and resistivity to intracellular conditions.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science and Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

### Peptides

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

The term “fragment” as used herein refers to a peptide having one or more deletions of amino acid residues relative to the sequences of the variant polypeptides listed herein, so long as the requisite activity is maintained. The amino acid residues may be deleted from the amino terminus and/or carboxy terminus and/or along the peptide sequence.

Peptide fragments can be produced by chemical synthesis, recombinant DNA technology, or by subjecting the peptides listed herein to at least one cleaving agent. A cleaving agent can be a chemical cleaving agent, e.g., cyanogen bromide, or an enzyme, e.g., an exoproteinase or endoproteinase.

Polypeptide products can be biochemically synthesized such as by employing standard solid phase techniques. Such methods include exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase polypeptide synthesis procedures are well known in the art and further described by John Morrow Stewart and Janis Dillaha Young, Solid Phase Peptide Syntheses (2nd Ed., Pierce Chemical Company, 1984).

Synthetic polypeptides can be purified by preparative high performance liquid chromatography (Creighton T. 1983 Proteins, structures and molecular principles. WH Freeman and Co. N.Y.) and the composition of which can be confirmed via amino acid sequencing.

In cases where large amounts of a polypeptide are desired, it can be generated using recombinant techniques such as described by Bitter et al., 1987 Methods in Enzymol. 153:516-544, Studier et al. 1990 Methods in Enzymol. 185:60-89, Brisson et al. 1984 Nature 310:511-514, Takamatsu et al. 1987 EMBO J. 6:307-311, Coruzzi et al. 1984 EMBO J. 3:1671-1680 and Brogli et al., 1984 Science 224:838-843, Gurley et al. 1986 Mol. Cell. Biol. 6:559-565 and Weissbach & Weissbach, 1988, Methods for Plant Molecular Biology, Academic Press, NY, Section VIII, pp 421-463.

It will be appreciated that peptides identified according to the teachings of the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH₂, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH₃)—CO—), ester bonds (—C(R)H—C—O—O—C(R)—N—), ketomethylen bonds (—CO—CH₂—), α-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Table 2 below lists non-conventional or modified amino acids which can be used with the present invention.

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorva1ine | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylva1ine | Mtrp | L-α-methyltyrosine | Mtyr |
| L-α-methylleucine | Mval | L-N-methylhomophenylalanine | Nmhphe |
| | nbhm | | |
| N-(N-(2,2-diphenylethyl) | Nnbhm | N-(N-(3,3-diphenylpropyl) | Nnbhe |
| carbamylmethyl-glycine | | carbamylmethyl(1)glycine | |
| 1-carboxy-1-(2,2-diphenyl | Nmbc | | |
| hylamino)cyclopropane | | | |

Since the peptides of the present invention are preferably utilized in therapeutics which requires the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention are preferably utilized in a cyclic form, although it will be appreciated that linear forms of the peptide can also be utilized.

The peptides of present invention can be biochemically synthesized such as by using standard solid phase techniques. These methods include exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography (Creighton T. 1983 Proteins, structures and molecular principles. WH Freeman and Co. N.Y.) and the composition of which can be confirmed via amino acid sequencing.

In cases where large amounts of the peptides of the present invention are desired, the peptides of the present invention can be generated using recombinant techniques such as described by Bitter et al., 1987 Methods in Enzymol. 153:516-544, Studier et al. 1990 Methods in Enzymol. 185:60-89, Brisson et al. 1984 Nature 310:511-514, Takamatsu et al. 1987 EMBO J. 6:307-311, Coruzzi et al. 1984 EMBO J. 3:1671-1680 and Brogli et al. 1984 Science 224:838-843, Gurley et al. 1986 Mol. Cell. Biol. 6:559-565 and Weissbach & Weissbach, 1988, Methods for Plant Molecular Biology, Academic Press, NY, Section VIII, pp 421-463.

### Expression Systems

To enable cellular expression of the polynucleotides of the present invention, a nucleic acid construct according to the present invention may be used, which includes at least a coding region of one of the above nucleic acid sequences, and further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any suitable promoter sequence can be used by the nucleic acid construct of the present invention.

Preferably, the promoter utilized by the nucleic acid construct of the present invention is active in the specific cell population transformed. Examples of cell type-specific and/or tissue-specific promoters include promoters such as albumin that is liver specific (Pinkert et al. 1987 Genes Dev. 1:268-277), lymphoid specific promoters (Calame et al. 1988 Adv. Immunol. 43:235-275); in particular promoters of T-cell receptors (Winoto et al. 1989 EMBO J. 8:729-733) and immunoglobulins; (Banerji et al. 1983 Cell 33:729-740), neuron-specific promoters such as the neurofilament promoter (Byrne et al. 1989 Proc. Natl. Acad. Sci. USA 86:5473-5477), pancreas-specific promoters (Edlunch et al. 1985 Science 230:912-916) or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the transgene is transcribed from CMV promoter. Vectors derived from Mo-MuLV are also included such as pBabe, where the transgene will be transcribed from the 5′LTR promoter.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol (Tonkinson et al. 1996 Cancer Investigation, 14(1): 54-65). The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

### Variant Recombinant Expression Vectors and Host Cells

Another aspect of the invention pertains to vectors, preferably expression vectors, containing a nucleic acid encoding a variant protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. 1990. Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., variant proteins, mutant forms of variant proteins, fusion proteins, etc.).

The recombinant expression vectors of the invention can be designed for production of variant proteins in prokaryotic or eukaryotic cells. For example, variant proteins can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, to the amino or C terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin, PreScission, TEV and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al. 1988 Gene 69:301-315) and pET 11d (Studier et al. Gene Expression Technology: Methods in Enzymology 185, Academic Press, San Diego, Calif. (1990) 60-89)—not accurate, pET11a-d have N terminal T7 tag.

One strategy to maximize recombinant protein expression in E. coli is to express the protein in a host bacterium with an impaired capacity to proteolytically cleave the recombinant protein. See, e.g., Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques. Another strategy to solve codon bias is by using BL21-codon plus bacterial strains (Invitrogen) or Rosetta bacterial strain (Novagen), these strains contain extra copies of rare E. coli tRNA genes.

In another embodiment, the expression vector encoding for the variant protein is a yeast expression vector. Examples of vectors for expression in yeast Saccharomyces cerivisae include pYepSec1 (Baldari, et al. 1987. EMBO J. 6:229-234), pMFa (Kurjan and Herskowitz, 1982 Cell 30:933-943), pJRY88 (Schultz et al., 1987 Gene 54:113-123), pYES2 (Invitrogen Corporation, San Diego, Calif.), and picZ (InVitrogen Corp, San Diego, Calif.).

Alternatively, variant protein can be produced in insect cells using baculovirus expression vectors. Baculovirus vectors available for expression of proteins in cultured insect cells (e.g., SF9 cells) include the pAc series (Smith, et al. 1983 Mol. Cell. Biol. 3:2156-2165) and the pVL series (Lucklow and Summers, 1989 Virology 170:31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al. 1987 EMBO J. 6: 187-195), pIRESpuro (Clontech), pUB6 (Invitrogen), pCEP4 (Invitrogen) pREP4 (Invitrogen), pcDNA3 (Invitrogen). When used in mammalian cells, the expression vector's control functions are often provided by viral regulatory elements. For example, commonly used promoters are derived from polyoma, adenovirus 2, cytomegalovirus, Rous Sarcoma Virus, and simian virus 40. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al. 1987 Genes Dev. 1:268-277), lymphoid-specific promoters (Calame and Eaton, 1988 Adv. Immunol. 43:235-275), in particular promoters of T cell receptors (Winoto and Baltimore, 1989 EMBO J. 8:729-733) and immunoglobulins (Banerji, et al. 1983 Cell 33:729-740; Queen and Baltimore, 1983 Cell 33:741-748), neuron-specific promoters (e.g., the neurofilament promoter; Byrne and Ruddle, 1989 Proc. Natl. Acad. Sci. USA 86:5473-5477), pancreas-specific promoters (Edlund, et al. 1985 Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990 Science 249:374-379) and the alpha-fetoprotein promoter (Campes and Tilghman, 1989 Genes Dev. 3:537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to mRNA encoding for variant protein. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, variant protein can be produced in bacterial cells such as E. coli, insect cells, yeast, plant or mammalian cells (such as Chinese hamster ovary cells (CHO) or COS or 293 cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals. For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin, puromycin, blasticidin and methotrexate. Nucleic acids encoding a selectable marker can be introduced into a host cell on the same vector as that encoding variant protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) variant protein. Accordingly, the invention further provides methods for producing variant protein using the host cells of the invention. In one embodiment, the method comprises culturing the host cell of the present invention (into which a recombinant expression vector encoding variant protein has been introduced) in a suitable medium such that variant protein is produced. In another embodiment, the method further comprises isolating variant protein from the medium or the host cell.

For efficient production of the protein, it is preferable to place the nucleotide sequences encoding the variant protein under the control of expression control sequences optimized for expression in a desired host. For example, the sequences may include optimized transcriptional and/or translational regulatory sequences (such as altered Kozak sequences).

### Protein Modifications

### Fusion Proteins

A fusion protein may be prepared from a variant protein according to the present invention by fusion with a portion of an immunoglobulin comprising a constant region of an immunoglobulin. More preferably, the portion of the immunoglobulin comprises a heavy chain constant region which is optionally and more preferably a human heavy chain constant region. The heavy chain constant region is most preferably an IgG heavy chain constant region, and optionally and most preferably is an Fc chain, most preferably an IgG Fc fragment that comprises CH2 and CH3 domains. Although any IgG subtype may optionally be used, the IgG1 subtype is preferred. The Fc chain may optionally be a known or “wild type” Fc chain, or alternatively may be mutated. Non-limiting, illustrative, exemplary types of mutations are described in US Patent Application No. 20060034852, published on Feb. 16, 2006, hereby incorporated by reference as if fully set forth herein. The term “Fc chain” also optionally comprises any type of Fc fragment.

Several of the specific amino acid residues that are important for antibody constant region-mediated activity in the IgG subclass have been identified. Inclusion, substitution or exclusion of these specific amino acids therefore allows for inclusion or exclusion of specific immunoglobulin constant region-mediated activity. Furthermore, specific changes may result in a glycosylation for example and/or other desired changes to the Fc chain. At least some changes may optionally be made to block a function of Fc which is considered to be undesirable, such as an undesirable immune system effect, as described in greater detail below.

Non-limiting, illustrative examples of mutations to Fc which may be made to modulate the activity of the fusion protein include the following changes (given with regard to the Fc sequence nomenclature as given by Kabat, from Kabat E A et al: Sequences of Proteins of Immunological Interest. US Department of Health and Human Services, NIH, 1991): 220C->S; 233-238 ELLGGP->EAEGAP; 265D->A, preferably in combination with 434N->A; 297N->A (for example to block N-glycosylation); 318-322 EYKCK->AYACA; 330-331AP->SS; or a combination thereof (see for example M. Clark, “Chemical Immunol and Antibody Engineering”, pp 1-31 for a description of these mutations and their effect). The construct for the Fc chain which features the above changes optionally and preferably comprises a combination of the hinge region with the CH2 and CH3 domains.

The above mutations may optionally be implemented to enhance desired properties or alternatively to block non-desired properties. For example, a glycosylation of antibodies was shown to maintain the desired binding functionality while blocking depletion of T-cells or triggering cytokine release, which may optionally be undesired functions (see M. Clark, “Chemical Immunol and Antibody Engineering”, pp 1-31). Substitution of 331 proline for serine may block the ability to activate complement, which may optionally be considered an undesired function (see M. Clark, “Chemical Immunol and Antibody Engineering”, pp 1-31). Changing 330 alanine to serine in combination with this change may also enhance the desired effect of blocking the ability to activate complement.

Residues 235 and 237 were shown to be involved in antibody-dependent cell-mediated cytotoxicity (ADCC), such that changing the block of residues from 233-238 as described may also blocks such activity if ADCC is considered to be an undesirable function.

Residue 220 is normally a cysteine for Fc from IgG1, which is the site at which the heavy chain forms a covalent linkage with the light chain. Optionally, this residue may be changed to a serine, to avoid any type of covalent linkage (see M. Clark, “Chemical Immunol and Antibody Engineering”, pp 1-31).

The above changes to residues 265 and 434 may optionally be implemented to reduce or block binding to the Fc receptor, which may optionally block undesired functionality of Fc related to its immune system functions (see “Binding site on Human IgG1 for Fc Receptors”, Shields et al. vol 276, pp 6591-6604, 2001).

The above changes are intended as illustrations only of optional changes and are not meant to be limiting in any way. Furthermore, the above explanation is provided for descriptive purposes only, without wishing to be bound by a single hypothesis.

### Addition of Groups

If a variant according to the present invention is a linear molecule, it is possible to place various functional groups at various points on the linear molecule which are susceptible to or suitable for chemical modification. Functional groups can be added to the termini of linear forms of the variant. In some embodiments, the functional groups improve the activity of the variant with regard to one or more characteristics, including but not limited to, improvement in stability, penetration (through cellular membranes and/or tissue barriers), tissue localization, efficacy, decreased clearance, decreased toxicity, improved selectivity, improved resistance to expulsion by cellular pumps, and the like. For convenience sake and without wishing to be limiting, the free N-terminus of one of the sequences contained in the compositions of the invention will be termed as the N-terminus of the composition, and the free C-terminal of the sequence will be considered as the C-terminus of the composition. Either the C-terminus or the N-terminus of the sequences, or both, can be linked to a carboxylic acid functional groups or an amine functional group, respectively.

Non-limiting examples of suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the active ingredient attached thereto into a cell, for example, by reducing the hydrophilicity and increasing the lipophilicity of the active ingredient, these being an example for “a moiety for transport across cellular membranes”.

These moieties can optionally and preferably be cleaved in vivo, either by hydrolysis or enzymatically, inside the cell. (Ditter et al., J. Pharm. Sci. 57:783 (1968); Ditter et al., J. Pharm. Sci. 57:828 (1968); Ditter et al., J. Pharm. Sci. 58:557 (1969); King et al., Biochemistry 26:2294 (1987); Lindberg et al., Drug Metabolism and Disposition 17:311 (1989); and Tunek et al., Biochem. Pharm. 37:3867 (1988), Anderson et al., Arch. Biochem. Biophys. 239:538 (1985) and Singhal et al., FASEB J. 1:220 (1987)). Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a composition of the present invention is protected, preferably with a methyl, ethyl, benzyl or substituted benzyl ester, more preferably as a benzyl ester.

Non-limiting, illustrative examples of N-terminal protecting groups include acyl groups (—CO—R1) and alkoxy carbonyl or aryloxy carbonyl groups (—CO—O—R1), wherein R1 is an aliphatic, substituted aliphatic, benzyl, substituted benzyl, aromatic or a substituted aromatic group. Specific examples of acyl groups include but are not limited to acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO— and (substituted benzyl)-CO—. Examples of alkoxy carbonyl and aryloxy carbonyl groups include CH3—O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, substituted phenyl-O—CO— and benzyl-O—CO—, (substituted benzyl)-O—CO—, Adamantan, naphtalen, myristoleyl, toluen, biphenyl, cinnamoyl, nitrobenzoy, toluoyl, furoyl, benzoyl, cyclohexane, norbornane, or Z-caproic. In order to facilitate the N-acylation, one to four glycine residues can be present in the N-terminus of the molecule.

The carboxyl group at the C-terminus of the compound can be protected, for example, by a group including but not limited to an amide (i.e., the hydroxyl group at the C-terminus is replaced with —NH₂, —NHR₂ and —NR₂R₃) or ester (i.e. the hydroxyl group at the C-terminus is replaced with —OR₂). R₂ and R₃ are optionally independently an aliphatic, substituted aliphatic, benzyl, substituted benzyl, aryl or a substituted aryl group. In addition, taken together with the nitrogen atom, R₂ and R₃ can optionally form a C4 to C8 heterocyclic ring with from about 0-2 additional heteroatoms such as nitrogen, oxygen or sulfur. Non-limiting suitable examples of suitable heterocyclic rings include piperidinyl, pyrrolidinyl, morpholino, thiomorpholino or piperazinyl. Examples of C-terminal protecting groups include but are not limited to —NH₂, —NHCH₃, —N(CH₃)₂, —NH(ethyl), —N(ethyl)₂, —N(methyl)(ethyl), —NH(benzyl), —N(C1-C4 alkyl)(benzyl), —NH(phenyl), —N(C1-C4 alkyl)(phenyl), —OCH₃, —O-(ethyl), —O-(n-propyl), —O-(n-butyl), —O-(iso-propyl), —O-(sec-butyl), —O-(t-butyl), —O-benzyl and —O-phenyl.

### Substitution by Peptidomimetic Moieties

A “peptidomimetic organic moiety” can optionally be substituted for amino acid residues in the composition of this invention both as conservative and as non-conservative substitutions. These moieties are also termed “non-natural amino acids” and may optionally replace amino acid residues, amino acids or act as spacer groups within the peptides in lieu of deleted amino acids. The peptidomimetic organic moieties optionally and preferably have steric, electronic or configurational properties similar to the replaced amino acid and such peptidomimetics are used to replace amino acids in the essential positions, and are considered conservative substitutions. However such similarities are not necessarily required. According to preferred embodiments of the present invention, one or more peptidonimetics are selected such that the composition at least substantially retains its physiological activity as compared to the native variant protein according to the present invention.

Peptidomimetics may optionally be used to inhibit degradation of the peptides by enzymatic or other degradative processes. The peptidomimetics can optionally and preferably be produced by organic synthetic techniques. Non-limiting examples of suitable peptidomimetics include D amino acids of the corresponding L amino acids, tetrazol (Zabrocki et al., J. Am. Chem. Soc. 110:5875-5880 (1988)); isosteres of amide bonds (Jones et al., Tetrahedron Lett. 29: 3853-3856 (1988)); LL-3-amino-2-propenidone-6-carboxylic acid (LL-Acp) (Kemp et al., J. Org. Chem. 50:5834-5838 (1985)). Similar analogs are shown in Kemp et al., Tetrahedron Lett. 29:5081-5082 (1988) as well as Kemp et al., Tetrahedron Lett. 29:5057-5060 (1988), Kemp et al., Tetrahedron Lett. 29:4935-4938 (1988) and Kemp et al., J. Org. Chem. 54:109-115 (1987). Other suitable but exemplary peptidomimetics are shown in Nagai and Sato, Tetrahedron Lett. 26:647-650 (1985); Di Maio et al., J. Chem. Soc. Perkin Trans., 1687 (1985); Kahn et al., Tetrahedron Lett. 30:2317 (1989); Olson et al., J. Am. Chem. Soc. 112:323-333 (1990); Garvey et al., J. Org. Chem. 56:436 (1990). Further suitable exemplary peptidomimetics include hydroxy-1,2,3,4-tetrahydroisoquinoline-3-carboxylate (Miyake et al., J. Takeda Res. Labs 43:53-76 (1989)); 1,2,3,4-tetrahydro-isoquinoline-3-carboxylate (Kazmierski et al., J. Am. Chem. Soc. 133:2275-2283 (1991)); histidine isoquinolone carboxylic acid (HIC) (Zechel et al., Int. J. Pep. Protein Res. 43 (1991)); (2S, 3S)-methyl-phenylalanine, (2S, 3R)-methyl-phenylalanine, (2R,3S)-methyl-phenylalanine and (2R, 3R)-methyl-phenylalanine (Kazmierski and Hruby, Tetrahedron Lett. (1991)).

Exemplary, illustrative but non-limiting non-natural amino acids include beta-amino acids (beta3 and beta2), homo-amino acids, cyclic amino acids, aromatic amino acids, Pro and Pyr derivatives, 3-substituted Alanine derivatives, Glycine derivatives, ring-substituted Phe and Tyr Derivatives, linear core amino acids or diamino acids. They are available from a variety of suppliers, such as Sigma-Aldrich (USA) for example.

### Chemical Modifications

In the present invention any part of a variant protein may optionally be chemically modified, i.e. changed by addition of functional groups. For example the side amino acid residues appearing in the native sequence may optionally be modified, although as described below alternatively other part(s) of the protein may optionally be modified, in addition to or in place of the side amino acid residues. The modification may optionally be performed during synthesis of the molecule if a chemical synthetic process is followed, for example by adding a chemically modified amino acid. However, chemical modification of an amino acid when it is already present in the molecule (“in situ” modification) is also possible.

The amino acid of any of the sequence regions of the molecule can optionally be modified according to any one of the following exemplary types of modification (in the peptide conceptually viewed as “chemically modified”). Non-limiting exemplary types of modification include carboxymethylation, acylation, phosphorylation, glycosylation or fatty acylation. Ether bonds can optionally be used to join the serine or threonine hydroxyl to the hydroxyl of a sugar. Amide bonds can optionally be used to join the glutamate or aspartate carboxyl groups to an amino group on a sugar (Garg and Jeanloz, Advances in Carbohydrate Chemistry and Biochemistry, Vol. 43, Academic Press (1985); Kunz, Ang. Chem. Int. Ed. English 26:294-308 (1987)). Acetal and ketal bonds can also optionally be formed between amino acids and carbohydrates. Fatty acid acyl derivatives can optionally be made, for example, by acylation of a free amino group (e.g., lysine) (Toth et al., Peptides: Chemistry, Structure and Biology, Rivier and Marshal, eds., ESCOM Publ., Leiden, 1078-1079 (1990)).

As used herein the term “chemical modification”, when referring to a protein or peptide according to the present invention, refers to a protein or peptide where at least one of its amino acid residues is modified either by natural processes, such as processing or other post-translational modifications, or by chemical modification techniques which are well known in the art. Examples of the numerous known modifications typically include, but are not limited to: acetylation, acylation, amidation, ADP-ribosylation, glycosylation, GPI anchor formation, covalent attachment of a lipid or lipid derivative, methylation, myristylation, pegylation, prenylation, phosphorylation, ubiquitination, or any similar process.

Other types of modifications optionally include the addition of a cycloalkane moiety to a biological molecule, such as a protein, as described in PCT Application No. WO 2006/050262, hereby incorporated by reference as if fully set forth herein. These moieties are designed for use with biomolecules and may optionally be used to impart various properties to proteins.

Furthermore, optionally any point on a protein may be modified. For example, pegylation of a glycosylation moiety on a protein may optionally be performed, as described in PCT Application No. WO 2006/050247, hereby incorporated by reference as if fully set forth herein. One or more polyethylene glycol (PEG) groups may optionally be added to O-linked and/or N-linked glycosylation. The PEG group may optionally be branched or linear. Optionally any type of water-soluble polymer may be attached to a glycosylation site on a protein through a glycosyl linker.

### Altered Glycosylation

Variant proteins of the invention may be modified to have an altered glycosylation pattern (i.e., altered from the original or native glycosylation pattern). As used herein, “altered” means having one or more carbohydrate moieties deleted, and/or having at least one glycosylation site added to the original protein.

Glycosylation of proteins is typically either N-linked or O-linked. N-linked refers to the attachment of the carbohydrate moiety to the side chain of an asparagine residue. The tripeptide sequences, asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to variant proteins of the invention is conveniently accomplished by altering the amino acid sequence of the protein such that it contains one or more of the above-described tripeptide sequences (for N-linked glycosylation sites). The alteration may also be made by the addition of, or substitution by, one or more serine or threonine residues in the sequence of the original protein (for O-linked glycosylation sites). The protein's amino acid sequence may also be altered by introducing changes at the DNA level.

Another means of increasing the number of carbohydrate moieties on proteins is by chemical or enzymatic coupling of glycosides to the amino acid residues of the protein. Depending on the coupling mode used, the sugars may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330, and in Aplin and Wriston, CRC Crit. Rev. Biochem., 22: 259-306 (1981).

Removal of any carbohydrate moieties present on variant proteins of the invention may be accomplished chemically or enzymatically. Chemical deglycosylation requires exposure of the protein to trifluoromethanesulfonic acid, or an equivalent compound. This treatment results in the cleavage of most or all sugars except the linking sugar (N-acetylglucosamine or N-acetylgalactosamine), leaving the amino acid sequence intact.

Chemical deglycosylation is described by Hakimuddin et al., Arch. Biochem. Biophys., 259: 52 (1987); and Edge et al., Anal. Biochem., 118: 131 (1981). Enzymatic cleavage of carbohydrate moieties on proteins can be achieved by the use of a variety of endo- and exo-glycosidases as described by Thotakura et al., Meth. Enzymol., 138: 350 (1987).

### Methods of Treatment

As mentioned hereinabove the novel therapeutic variants of the present invention and compositions derived therefrom (i.e., proteins, peptides, oligonucleotides) can be used to treat cluster or protein-related diseases, disorders or conditions.

Thus, according to an additional aspect of the present invention there is provided a method of treating cluster or ANP-related disease, disorder or condition in a subject . . .

The subject according to the present invention is a mammal, preferably a human which is diagnosed with one of the disease, disorder or conditions described hereinabove, or alternatively is predisposed to at least one type of the cluster or protein-related disease, disorder or conditions described hereinabove.

As used herein the term “treating” refers to preventing, curing, reversing, attenuating, alleviating, minimizing, suppressing or halting the deleterious effects of the above-described diseases, disorders or conditions.

Treating, according to the present invention, can be effected by specifically upregulating or alternatively downregulating the expression of at least one of the polypeptides of the present invention in the subject.

Optionally, upregulation may be effected by administering to the subject at least one of the polypeptides of the present invention (e.g., recombinant or synthetic) or an active portion thereof, as described herein. However, since the bioavailability of large polypeptides may potentially be relatively small due to high degradation rate and low penetration rate, administration of polypeptides is preferably confined to small peptide fragments (e.g., about 100 amino acids). The polypeptide or peptide may optionally be administered in as part of a pharmaceutical composition, described in more detail below.

It will be appreciated that treatment of the above-described diseases according to the present invention may be combined with other treatment methods known in the art (i.e., combination therapy). Thus, treatment of malignancies using the agents of the present invention may be combined with, for example, radiation therapy, antibody therapy and/or chemotherapy.

Alternatively or additionally, an upregulating method may optionally be effected by specifically upregulating the amount (optionally expression) in the subject of at least one of the polypeptides of the present invention or active portions thereof.

As is mentioned hereinabove and in the Examples section which follows, the biomolecular sequences of this aspect of the present invention may be used as valuable therapeutic tools in the treatment of diseases, disorders or conditions in which altered activity or expression of the wild-type gene product is known to contribute to disease, disorder or condition onset or progression. For example, in case a disease is caused by overexpression of a membrane bound-receptor, a soluble variant thereof may be used as an antagonist which competes with the receptor for binding the ligand, to thereby terminate signaling from the receptor. Examples of such diseases are listed in the Examples section which follows.

It will be appreciated that the polypeptides of the present invention may also have agonistic properties. These include increasing the stability of the ligand (e.g., IL-4), protection from proteolysis and modification of the pharmacokinetic properties of the ligand (i.e., increasing the half-life of the ligand, while decreasing the clearance thereof). As such, the biomolecular sequences of this aspect of the present invention may be used to treat conditions or diseases in which the wild-type gene product plays a favorable role, for example, increasing angiogenesis in cases of diabetes or ischemia.

Upregulating expression of the therapeutic protein or polypeptide variants of the present invention may be effected via the administration of at least one of the exogenous polynucleotide sequences of the present invention, ligated into a nucleic acid expression construct (as described in greater detail hereinabove) designed for expression of coding sequences in eukaryotic cells (e.g., mammalian cells), as described above. Accordingly, the exogenous polynucleotide sequence may be a DNA or RNA sequence encoding the variants of the present invention or active portions thereof.

It will be appreciated that the nucleic acid construct can be administered to the individual employing any suitable mode of administration including in vivo gene therapy (e.g., using viral transformation as described hereinabove). Alternatively, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy).

Such cells (i.e., which are transfected with the nucleic acid construct of the present invention) can be any suitable cells, such as kidney, bone marrow, keratinocyte, lymphocyte, adult stem cells, cord blood cells, embryonic stem cells which are derived from the individual and are transfected ex vivo with an expression vector containing the polynucleotide designed to express the polypeptide of the present invention as described hereinabove.

Administration of the ex vivo transfected cells of the present invention can be effected using any suitable route such as intravenous, intra peritoneal, intra kidney, intra gastrointestinal track, subcutaneous, transcutaneous, intramuscular, intracutaneous, intrathecal, epidural and rectal. According to presently preferred embodiments, the ex vivo transfected cells of the present invention are introduced to the individual using intravenous, intra kidney, intra gastrointestinal track and/or intra peritoneal administrations.

The ex vivo transfected cells of the present invention can be derived from either autologous sources such as self bone marrow cells or from allogeneic sources such as bone marrow or other cells derived from non-autologous sources. Since non-autologous cells are likely to induce an immune reaction when administered to the body several approaches have been developed to reduce the likelihood of rejection of non-autologous cells. These include either suppressing the recipient immune system or encapsulating the non-autologous cells or tissues in immunoisolating, semipermeable membranes before transplantation.

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamylidene-acetylated poly(allylamine). Biotechnol Bioeng. 2000, 70: 479-83, Chang T M and Prakash S. Procedures for microencapsulation of enzymes, cells and genetically engineered microorganisms. Mol. Biotechnol. 2001, 17: 249-60, and Lu M Z, et al., A novel cell encapsulation method using photosensitive poly(allylamine alpha-cyanocinnamylideneacetate). J Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Thechnol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002; 13: 783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

It will be appreciated that the present methodology may also be effected by specifically upregulating the expression of the variants of the present invention endogenously in the subject. Agents for upregulating endogenous expression of specific splice variants of a given gene include antisense oligonucleotides, which are directed at splice sites of interest, thereby altering the splicing pattern of the gene. This approach has been successfully used for shifting the balance of expression of the two isoforms of Bcl-x (Taylor 1999 Nat. Biotechnol. 17:1097-1100; and Mercatante 2001 J. Biol. Chem. 276:16411-16417); IL-5R (Karras 2000 Mol. Pharmacol. 58:380-387); and c-myc (Giles (1999) Antisense Acid Drug Dev. 9:213-220).

For example, interleukin 5 and its receptor play a critical role as regulators of hematopoiesis and as mediators in some inflammatory diseases such as allergy and asthma. Two alternatively spliced isoforms are generated from the IL-5R gene, which include (i.e., long form) or exclude (i.e., short form) exon 9. The long form encodes for the intact membrane-bound receptor, while the shorter form encodes for a secreted soluble non-functional receptor. Using 2′-O-MOE-oligonucleotides specific to regions of exon 9, Karras and co-workers (supra) were able to significantly decrease the expression of the wild type receptor and increase the expression of the shorter isoforms. Design and synthesis of oligonucleotides which can be used according to the present invention are described hereinbelow and by Sazani and Kole (2003) Progress in Molecular and Subcellular Biology 31:217-239.

### Pharmaceutical Compositions and Delivery Thereof.

The present invention features a pharmaceutical composition comprising a therapeutically effective amount of a therapeutic agent according to the present invention, which is preferably a therapeutic protein variant as described herein. Optionally and alternatively, the therapeutic agent could be an antibody or an oligonucleotide that specifically recognizes and binds to the therapeutic protein variant, but not to the corresponding full length known protein.

According to the present invention the therapeutic agent could be any one of novel ANP variant polypeptides and polynucleotides of the present invention. Optionally and alternatively, the therapeutic agent could be an antibody or an oligonucleotide that specifically recognizes and binds to the novel ANP variant polypeptides and polynucleotides of the present invention.

According to the present invention the therapeutic agent could be used for the treatment or prevention of a wide range of diseases, including but not limited to cardiovascular diseases, such as heart failure, stroke, renal failure, sudden cardiac death from arrhythmia or any other heart related reason, rejection of a transplanted heart, conditions that lead to heart failure including but not limited to myocardial infarction, angina, arrhythmias, valvular diseases, conditions that cause atrial and or ventricular wall volume overload, systemic arterial hypertension, pulmonary hypertension and pulmonary embolism.

Alternatively, the pharmaceutical composition of the present invention includes a therapeutically effective amount of at least an active portion of a therapeutic protein variant polypeptide.

The pharmaceutical composition according to the present invention is preferably used for the treatment of cluster-related diseases.

“Treatment” refers to both therapeutic treatment and prophylactic or preventative measures. Those in need of treatment include those already with the disorder as well as those in which the disorder is to be prevented. Hence, the mammal to be treated herein may have been diagnosed as having the disorder or may be predisposed or susceptible to the disorder. “Mammal” for purposes of treatment refers to any animal classified as a mammal, including humans, domestic and farm animals, and zoo, sports, or pet animals, such as dogs, horses, cats, cows, etc. Preferably, the mammal is human.

A “disorder” is any condition that would benefit from treatment with the agent according to the present invention. This includes chronic and acute disorders or diseases including those pathological conditions which predispose the mammal to the disorder in question. Non-limiting examples of disorders to be treated herein are described with regard to specific examples given herein.

The term “therapeutically effective amount” refers to an amount of agent according to the present invention that is effective to treat a disease or disorder in a mammal. In the case of cancer, the therapeutically effective amount of the agent may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the agent may prevent growth and/or kill existing cancer cells, it may be cytostatic and/or cytotoxic. For cancer therapy, efficacy can, for example, be measured by assessing the time to disease progression (TTP) and/or determining the response rate (RR).

The therapeutic agents of the present invention can be provided to the subject per se, or as part of a pharmaceutical composition where they are mixed with a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the preparation accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections. Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Pharmaceutical compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Cleavage Sites Prediction

Many secretory proteins are synthesized as inactive precursors that must undergo post-translational proteolysis to become biologically active polypeptides. It is often desirable to know the exact sequence of mature proteins and peptides, but this is experimentally hard to achieve. Moreover, current computational approaches are limited to specific proteolytic enzymes. In order to predict cleavage sites in secreted and extracellular domains of membrane proteins various classifiers were trained on experimental data extracted from the Swiss-Prot protein database. This method is described in greater detail below (under “Methods” paragraph below). The classifiers that were the most successful methods for discriminating between proteolytic cleavage sites and non-cleavage sites were those based on Breiman's random forest (Breiman, L. and A. Cutler Random Forests).

An important finding is the estimated number of cleavage sites, currently not annotated as such. Herein, we focused on proteolytic sites occurring immediately after lysines or arginines. There are 468 such proteolytic sites (VALIDATED & POTENTIAL, see below), and 860 unknown (“hidden” in NON & AMBG, see below). This means that the actual number of such proteolytic sites is about 2.84 times the currently known number. In addition we have obtained an estimate for the proportion of proteolytic sites in the set of all sites after arginine or lysine—about 1%. In other words, many proteolytic sites are currently not annotated as cleavage sites, but are defined as such by our classifier with a high probability. These include proteins that are in development as therapeutic proteins, and in a few cases experiments aiming to find the minimal sequence required for functionality identify these exact, potentially cleaved peptides, as the minimum required sequence. One of the predicted cleavage sites with a very high score is in the newly discovered splice variant of ANP according to the present invention, as described below.

### Methods

All mammalian proteins were downloaded from three versions of the Swiss-Prot knowledgebase: version 39.0, version 42.0, and version 47.4. Proteins whose first residue is not methionine were discarded as they might not contain the sequence of the full-length precursor protein. The same holds for proteins whose RP annotation lines contain only “PROTEIN SEQUENCE”, but not “NUCLEOTIDE SEQUENCE”, as these may be processed proteins, and not the full-length precursor proteins. Data of proteolytic cleavage sites was extracted from the post-translational modifications annotation lines of the Swiss-Prot knowledgebase (Farriol-Mathis, N., J. S. Garavelli, et al. 2004 Proteomics 4(6): 1537-50).

The aim of the classifier was to model the proteolytic processes that take place in the secretory pathway; therefore only secreted proteins and extracellular parts of membranal proteins (secretome) were considered. Thus, only proteins with annotation for signal peptide in the first FT line of the Swiss-Prot annotation record, or annotation for a secreted or extracellular protein in the CC lines of the SwissProt annotation record, were selected. For integral membrane proteins, the cytoplasmic domains were not considered. When the topology annotation lines (FT TOPO_DOM and FT TRANSMEM) of the SwissProt annotation record did not comprise the full length of the protein, it was completed according to the annotated signal peptide, transmembrane domains, extracellular domains, and cytoplasmic domains. This process was performed twice—once by starting from the most N-terminus topology annotation, and once by starting from the most C-terminus topology annotation. When discrepancies between the two completion processes were found, the protein was discarded. This may happen in multi-span proteins.

Currently, most computational approaches are protease-oriented, i.e. the cleavage site data they rely on is of specific enzymes (Yang, Z. R. and E. A. Berry 2004 J Bioinform Comput Biol 2(3):511-31; Kiemer, L., O. Lund, et al. 2004 BMC Bioinformatics 5:72; Cai, Y. D., H. Yu, et al. 1998 Protein Sci 5(11):2203-16.). However, the amount of experimentally verified sites, where the bona fide proteolytic enzyme is known, is still limited. This stems from the fact that it is much easier to identify the site of cleavage in a protein, usually done by N-terminal sequencing, than to identify the nature of the physiologically active proteolytic enzyme. Thus, only a limited number of experimentally verified cleavage sites can be extracted for most proteolytic enzymes. Herein, all proteolytic cleavage sites that could be extracted from Swiss-Prot were used, even when no enzyme information is available.

The extracted proteolytic cleavage sites should be divided to those catalyzed by enzymes working in the secretory pathway, the extracellular matrix, the cytoplasm, the digestion system, or in extracellular fluids. As the aim of this study is to model the processes that take place in the secretory pathways, annotation of the proteolytic enzyme was extracted from the FT annotation lines (after “Removed by” in the description of PROPEPs lines, or after “by” in the description of SITE . . . CLEAVAGE lines), where found. Proteolytic cleavage sites processed by enzymes that are known to act outside the secretory pathway were discarded. The list of enzymes known to act outside the secretory pathway which appear in Swiss-Prot annotations for the proteins that they cleave includes but is not limited to: adam17, aggrecanase, alpha-secretase, beta-secretase, caspase-6, cathepsin G, arginine-specific endoprotease, C3 convertase, chymosin, collagenase, dipeptidase, dipeptidylpeptidase, DPP4, easter, elastase, kallikrein and kallikrein-like serine protease, MMPs (2, 3, and 9), coagulation factors (I, VIIa, IXa, Xa, and XIa), plasmin, procollagen C-endopeptidase, procollagen N-endopeptidase, rennin, thrombin, trypsin, and u-PA.

The aim was to build a classifier that predicts proteolytic cleavage sites of enzymes that cut after lysine and arginine residues. Such enzymes are often classified as members of the pro-hormone convertase family. Therefore, only cleavage sites with a lysine or arginine at the first position N-terminal to the proteolytic cleavage site were considered. We extracted all 30-mers of the secretome and labeled them as follows:

(1) A position with no annotation was designated as NON.

(2) An experimentally-validated proteolytic cleavage site that is annotated by a Swiss-Prot FT annotation line according to the word template SITE . . . CLEAVAGE was designated as VALIDATED.

(3) An experimentally-validated proteolytic cleavage site whose existence is supported by two Swiss-Prot FT annotation lines having the word template “PEPTIDE, PROPEPTIDE, or CHAIN [first residue] [last residue]”, and the two segments of the protein are consecutive, was designated as VALIDATED. By consecutive we mean that the first residue of the second segment immediately follows the last residue of the first segment. We also allow a short sequence between the two segments, but only if it is likely that exopeptidase E removes these residues after the processing of the protein precursor by a pro-hormone convertase (Friis-Hansen, L., K. A. Lacourse, et al. 2001 J Endocrinol 169(3):595-602; Day, R., C. Lazure, et al. 1998 J Biol Chem 273(2):829-36).

Cases that comprised of K, R, KK, KR, RK, RR, sequence of Ks and/or Rs that is finished by a classical furin cleavage site (RXKR or RXRR, where X is any natural amino acid) were considered as likely to be cut by exopeptidase E. A glycine as the first residue after the first PEPTIDE, PROPEPTIDE, or CHAIN, was allowed, as it is likely that these peptides are substrates for C-terminal alpha-amidating enzymes that convert the peptides to the corresponding des-glycine peptide amide, and the glycine is the amide donor (Bradbury, A. F., M. D. Finnie, et al. 1982 Nature 298(5875):686-8). The sites after the residues that fall between the two annotation lines are designated as AMBG.

(4) When only one PEPTIDE, PROPEPTIDE, or CHAIN annotation line suggests a proteolytic cleavage site, the site of proteolysis is of a lesser degree of confidence, thus these sites are designated as POTENTIAL.

(5) When comments like ‘PROBABLE’, ‘BY SIMILARITY’, or ‘POTENTIAL’ (Junker, V. L., R. Apweiler, et al. 1999, Bioinformatics 15(12):1066-7; Friis-Hansen, L., K. A. Lacourse, et al. 2001 J Endocrinol 169(3):595-602; Farriol-Mathis, N., J. S. Garavelli, et al. 2004 Proteomics 4(6):1537-50) appear in the description of the FT lines in the cases described in (2) and (3), the cleavage sites were designated as POTENTIAL.

(6) When two cleavage sites are at a distance of no more than 4 residues their reliability is diminished. Therefore, such cleavage sites are designated POTENTIAL unless there is other strong support for their reliability. By strong support it is meant that the proteolytic site is designated as VALIDATED through the annotation record according to (2); or designated POTENTIAL according to the SITE . . . CLEAVAGE annotation line of the annotation record & VALIDATED through this record according to (3).

For training only reliable proteolytic cleavage sites were used. Thus, for training, only proteolytic sites that were designated VALIDATED were labeled as positives, and those designated NON were labeled as negatives.

For testing it is important that all data be used for performance evaluation. Thus, proteolytic sites that were designated VALIDATED or POTENTIAL were labeled as positives, and those designated NON or AMBG were labeled as negatives.

One of the critical issues in developing a classifier was generalization. Thus, the classifier might be fed all known data available up to a given point in time, and tested on all data “discovered in the future”. Another point that may affect the generalization of a classifier is parameter optimization. In order to avoid the risk of overfitting, parameters were optimized on a separate validation set. The classifier was built in two steps:

(1) Parameter tuning. Proteolytic cleavage sites data extracted from Swiss-Prot version 39.0 was used for training, and data from Swiss-Prot version 42.0, that does not appear in Swiss-Prot version 39.0, was used for testing. Precision vs. recall graphs were evaluated in order to find optimal settings, using the raw score output of random forest. Several parameters were thus optimized. A symmetrical window of 18 residues around a site was found to be adequate. A negative set 30 times larger than the positive set was found to be best. This was done in combination with the internal weighting mechanism of Random Forest, set to give the two sets equal weights. Mtry was set to 3, and finally, 500 trees were found to be sufficient.

(2) Classifier construction and performance evaluation. Data from Swiss-Prot version 42.0 was used for training, and the data from Swiss-Prot version 47.4 that does not appear in Swiss-Prot version 42.0 was used for testing. The parameters used were those found to be optimal in step (1).

### Performance Evaluation:

As explained in the previous section, all data in the test set was included, in order to reflect the characteristics of all available data. In some cases there was uncertainty about the label of any data that is not validated positive: sites designated POTENTIAL and AMBG may or may not be real proteolytic sites, and proteolytic sites, not yet discovered, are hidden in the sites designated NON.

Recall=(TPi+TPo)/(Ti+To) (i)

Where, TP—true positives, T—what is really true, i—positives, o—negatives.

Precision=(TPi+TPo)/(Pi+Po) (ii)

Where P is what the classifier predicts to be true.

When precision vs recall graphs were drown, the results were obviously wrong: for a given threshold, the calculated recall did not include the mislabeled positives, so the precision evaluations were always underestimations. The reason was that while the denominator of equation (ii) is true, the numerator is smaller than it really is. There was no point in handling the POTENTIALs separately, since at least a portion of both the POTENTIALs and the NONs were likely to be mislabeled, even if the extent is different.

The following model was used. It was assumed that negative data is a mixture of two statistical types of data—mislabeled positives (proportion a of the negative data) and real negatives. The former has the same statistical nature as positive data.

Let Fi (Fo) be the cumulative distribution function of the score in positive (negative) data.

Let Ni (No) be the number of positive (negative) instances.

Let t be a threshold for the score.

Recall=(Ni(1−Fi(t))+αNo(1−Fi(t)))/(Ni+αNo)=1−Fi(t)

Note that recall does not depend on α, so it is equal to ordinary recall calculated without assuming any mislabeling.

Precision=(Ni(1−Fi(t))+αNo(1−Fi(t)))/(Pi+Po)=(1+αNo/Ni)*Ni(1−Fi(t))/(Pi+Po)

If α=0 we get Ni(1−Fi(t))/(Pi+Po). Therefore, the real precision is ordinary precision multiplied by a correction factor—(1+αNo/Ni).

In summary, in the mixture model, mislabeling leaves recall unchanged, and precision is multiplied by (1+αNo/Ni)=1+To/Ti.

For Furin proteolysis, there was a good estimate for this factor, because Furin sites have an easily detectable consensus, which can be used to calculate the above correction factor. Extrapolation was made from Furin to proteolytic sites of other members of the pro-hormone convertase family proteolysis, reflecting the curation level of proteolysis annotation in the Swiss-Prot knowledgebase.

Furin proteolysis consensus site, after RXKR or after RXRR, were searched in the positive and negative sets.

The instances in the positive set were real positives, whereas the ones in the negative set were a mixture of cleavage and non-cleavage sites.

There is evidence that a lysine two positions after the putative cleavage sites prevents cleavage, so such instances were excluded. In addition, it was observed which residues are most frequent immediately after the cleavage site in the positive set. The strategy for finding mislabeled cleavage sites in the negative set was as follows:

Instances of a Furin consensus followed by one of the 3 most frequent residues (as found in the positive set) were found, excluding lysines in the second post-cleavage position. The appropriate instances in the negative set, and exactly such instances in the positive set were counted, and the desired ratio followed, 2.84 in this case.

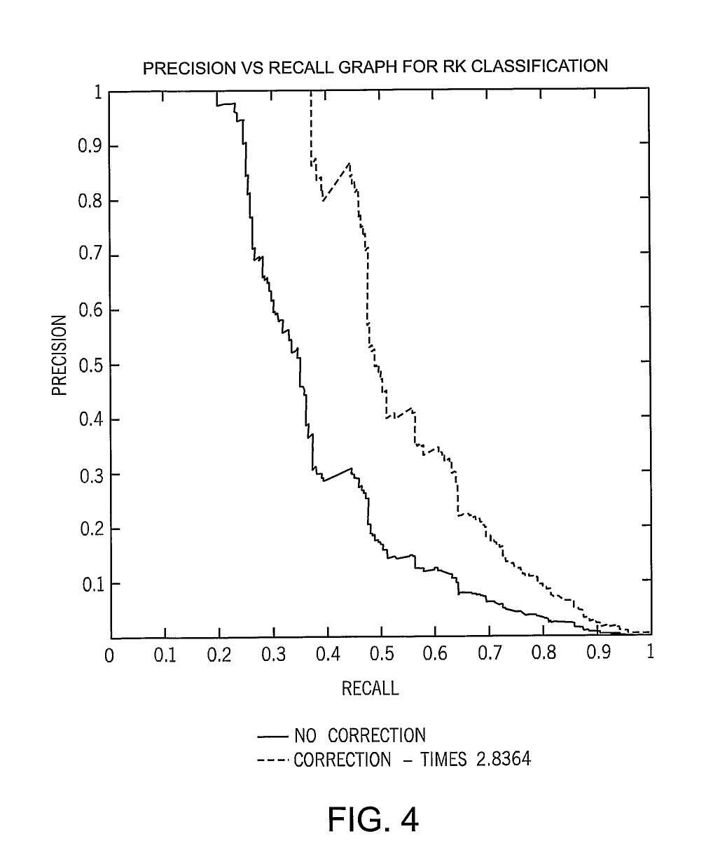

The graph presented in FIG. 4 shows the performance of a classifier trained on Swiss-Prot version 42 and tested on version 47.4.

FIG. 4 demonstrates precision vs. recall graphs. Validated and Potential data are treated as positive for testing, the rest as negative. The Furin correction is a way to compensate for the fact that some of the data treated as negatives for cleavage was actually mislabeled (unknown cleavage sites). The solid-line graph is calculated from the raw data, and the dashed-line graph is after taking into account the Furin correction factor.

The prediction for proteolytic cleavage sites for ANP variants is shown in Table 3. Table 4 describes the precision of each predicted cleavage site using the above-described methodology. The 1st column describes the protein name and its respective SEQ ID NO. The 2nd column indicates the cleavage site position on the precursor protein's sequence. The 3rd and the 4th columns indicate cleavage prediction raw score and precision calibrated score of prediction, respectively.

TABLE 3
Precision of cleavage sites

| | | | | Precision- |
|---|---|---|---|---|
| | SEQ | After | | calibrated |
| Protein Name | ID NO: | residue | Raw score | score |
| ANF_HUMAN | 13 | 123 | 27.694000 | 1.0 |
| ANF_HUMAN | 13 | 126 | 2.563000 | 5.0 |
| ANF_HUMAN | 13 | 127 | 1.501000 | 7.0 |
| NP_006163 | 14 | 123 | 27.694000 | 1.0 |
| NP_006163 | 14 | 126 | 2.563000 | 5.0 |
| NP_006163 | 14 | 127 | 1.501000 | 7.0 |
| HUMCDDANF_1_P4 | 15 | 123 | 27.694000 | 1.0 |
| HUMCDDANF_1_P4 | 15 | 126 | 2.563000 | 5.0 |
| HUMCDDANF_1_P4 | 15 | 127 | 1.501000 | 7.0 |
| HUMCDDANF_1_P5_{—} | 16 | 123 | 27.694000 | 1.0 |
| HUMCDDANF_1_P5_{—} | 16 | 126 | 2.563000 | 5.0 |
| HUMCDDANF_1_P5_{—} | 16 | 127 | 1.501000 | 7.0 |

Precision-calibrated score is from 1 to 10, reflecting precision intervals of 90-100% to 0-10%, respectively.

Thus, the above classifier has a high probability of correctly selecting cleavage sites in proteins, including new (not previously discovered) cleavage sites. This classifier and method was used to generate the ANP cleavage peptides described herein (see Example 4 herein).

### Example 2

### Description for Cluster HUMCDD_ANF

Cluster HUMCDD_ANF features 2 transcripts HUMCDDANF_{—}1_T3 (SEQ ID NO:1) and HUMCDDANF_{—}1T4 (SEQ ID NO:2); and 10 segments of interest, the names of which are given in Table 4. The selected protein variants are given in table 5.

TABLE 4
Segments of interest

| Segment Name | SEQ ID NO: |
|---|---|
| HUMCDDANF_1_N2 | 3 |
| HUMCDDANF_1_N5 | 4 |
| HUMCDDANF_1_N7 | 5 |
| HUMCDDANF_1_N8 | 6 |
| HUMCDDANF_1_N9 | 7 |
| HUMCDDANF_1_N13 | 8 |
| HUMCDDANF_1_N3 | 9 |
| HUMCDDANF_1_N6 | 10 |
| HUMCDDANF_1_N10 | 11 |
| HUMCDDANF_1_N11 | 12 |

TABLE 5
Proteins of interest

| Protein | Corresponding | Corresponding |
|---|---|---|
| Name | Transcript(s) | Peptides |
| HUMCDDANF_1_P4 | HUMCDDANF_1_T3 | 2A, 2B, 2C, 2D, 3A, |
| (Variant 2) | (SEQ ID NO:1) | 3B, 3C, 3D, 4A, 4B, |
| (SEQ ID NO:15) | | 4C, 4D |
| | | (SEQ ID NOs:25-36) |
| HUMCDDANF_1_P5 | HUMCDDANF_1_T4 | 1A, 1B, 1C, 1D |
| (Variant 1) | (SEQ ID NO:2) | (SEQ ID NOs:21-24) |
| (SEQ ID NO:16) | | |

These sequences are variants of the known protein Atrial natriuretic factor precursor (SwissProt accession identifier ANF_HUMAN (SEQ ID NO:13) known also according to the synonyms ANF; Atrial natriuretic peptide; ANF; Prepronatriodilatin; referred to herein as the previously known protein, which is believed to be secreted protein.

Known polymorphisms for this sequence are as shown in Table 6.

TABLE 6
Amino acid mutations for Known Protein

| SNP position(s) on | |
|---|---|
| amino acid sequence | Comment |
| 32 | V -> M (in dbSNP:5063). /FTId = VAR_014579 |
| 152-153 | Missing (in isoform 2). /FTId = VAR_000594 |
| 65 | E -> D |

Several ANP variants (HUMCDDANF_{—}1_P4 (SEQ ID NO:15), also called “variant 2”, described in PCT/IL2006/000676 to the applicant of the present invention and novel ANP variant HUMCDDANF_{—}1_P5 (SEQ ID NO:16), also called “variant 1”) were computationally identified, and predicted to function as natural natriuretic peptide receptor A (NPR-A) agonists and to have a therapeutic potential in CHF. The discovery was achieved through the use of several proprietary platforms, including the LEADS™ system (as described hereinabove) which has a proven track record of accurately predicting natural splice variants. PCT/IL2006/000676 and WO05069724 to the applicant of the present invention disclose the HUMCDDANF_{—}1_P4 variant and other ANP variants for use in diagnostic for the detection of cardiac disease and/or pathology and/or condition and/or disorder selected from the group consisting of Myocardial infarct, acute coronary syndrome, angina pectoris (stable and unstable), cardiomyopathy, myocarditis, congestive heart failure or any type of heart failure, the detection of reinfarction, the detection of success of thrombolytic therapy after Myocardial infarct, Myocardial infarct after surgery, assessing the size of infarct in Myocardial infarct, the differential diagnosis of heart related conditions from lung related conditions (as pulmonary embolism), the differential diagnosis of Dyspnea, and cardiac valves related conditions.

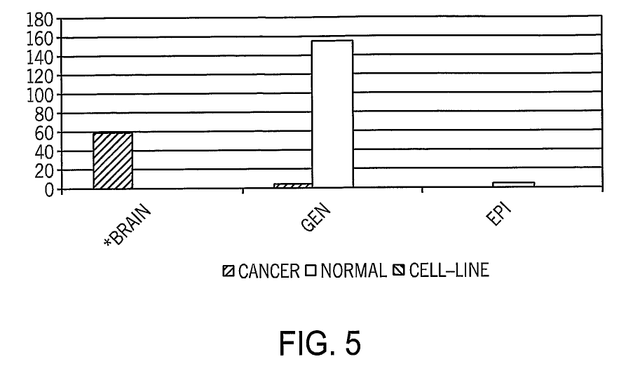

The transcripts of cluster HUMCDD_ANF are overexpressed in cancer, as is shown in Table 8 hereinbelow, which provides P value and ratios of the transcripts expression in cancerous tissue. Expression of such transcripts in normal tissues is also given according to the previously described methods (Table 7). The term “number” in the right hand column of table 7 and the numbers on the y-axis of FIG. 5 refer to weighted expression of ESTs in each category, as “parts per million” (ratio of the expression of ESTs for a particular cluster to the expression of all ESTs in that category, according to parts per million).

This cluster is particularly overexpressed (at least at a minimum level) in brain malignant tumors.

TABLE 7
Normal tissue distribution

| Name of Tissue | Number |
|---|---|
| brain | 0 |
| general | 154 |
| epithelial | 4 |

TABLE 8
P values and ratios for expression in cancerous tissue

| Name of Tissue | P1 | P2 | SP1 | R3 | SP2 | R4 |
|---|---|---|---|---|---|---|
| brain | 5.3e−02 | 9.5e−02 | 4.0e−04 | 11.6 | 6.9e−03 | 5.8 |
| general | 9.6e−01 | 9.8e−01 | 1.0e+00 | 0.0 | 1.0e+00 | 0.0 |
| epithelial | 8.9e−01 | 9.6e−01 | 1.0e+00 | 0.4 | 1.0e+00 | 0.3 |

As noted above, cluster HUMCDD_ANF features 2 transcripts that encode for proteins which are variants of protein Atrial natriuretic factor precursor. A description of each variant protein according to the present invention is presented hereinabove.

Variant protein HUMCDDANF_{—}1_P4 (SEQ ID NO:15) is encoded by transcript HUMCDDANF_{—}1_T3 (SEQ ID NO:1). An alignment of the variant protein to the known atrial natriuretic factor precursor is given in FIG. 6. A Comparison between HUMCDDANF_{—}1_P4 (SEQ ID NO:15) and ANF_HUMAN (SEQ ID NO:13), is shown in FIG. 6A. A Comparison between HUMCDDANF_{—}1_P4 (SEQ ID NO:15) and NP_{—}006163 (SEQ ID NO:14), is shown in FIG. 6B. A brief description of the relationship of the variant protein to each such aligned protein is as follows:

A. The isolated chimeric polypeptide designated HUMCDDANF_{—}1_P4 (SEQ ID NO:15), comprises a first amino acid sequence being at least 90% homologous to MSSFSTTTVSFLLLLAFQLLGQTRANPMYNAVSNADLMDFKNLLDHLEEKMPLE DEVVPPQVLSEPNEEAGAALSPLPEVPPWTGEVSPAQRDGGALGRGPWDSSDRS ALLKSKLRALLTAPRSLRRSSCFGGRMDRIGAQSGLGCNSFR corresponding to amino acids 1-150 of ANF_HUMAN (SEQ ID NO:13) or NP_{—}006163 (SEQ ID NO:14), which also corresponds to amino acids 1-150 of HUMCDDANF_{—}1_P4 (SEQ ID NO:15), and a second amino acid sequence being at least 70%, optionally at least 80%, preferably at least 85%, more preferably at least 90% and most preferably at least 95% homologous to a polypeptide having the sequence VRGTGDGNGMGWTLLGDTFSRKGTNAEAHSLSSFCPNTQSAPWVSGHAIYCP (SEQ ID NO:39) corresponding to amino acids 151-202 of HUMCDDANF_{—}1_P4 (SEQ ID NO:15), wherein said first amino acid sequence and second amino acid sequence are contiguous and in a sequential order.

B. An isolated polypeptide designated an edge portion of HUMCDDANF_{—}1_P4 (SEQ ID NO:15), comprises an amino acid sequence being at least 70%, optionally at least about 80%, preferably at least about 85%, more preferably at least about 90% and most preferably at least about 95% homologous to the sequence VRGTGDGNGMGWTLLGDTFSRKGTNAEAHSLSSFCPNTQSAPWVSGHAIYCP (SEQ ID NO:39) of HUMCDDANF_{—}1_P4 (SEQ ID NO:15).

C. A bridge portion of HUMCDDANF_{—}1_P4 (SEQ ID NO:15) comprises a polypeptide having a length “n”, wherein n is at least about 10 amino acids in length, optionally at least about 15 amino acids in length, preferably at least about 20 amino acids in length, more preferably at least about 25 amino acids in length and most preferably at least about 30 amino acids in length, wherein at least two amino acids comprise RV, having a structure as follows (numbering according to HUMCDDANF_{—}1_P4 (SEQ ID NO:15)): a sequence starting from any of amino acid numbers 150-x to 150; and ending at any of amino acid numbers 151+((n−2)−x), in which x varies from 0 to n−2.

The location of the variant protein was determined according to results from a number of different software programs and analyses, including analyses from SignalP and other specialized programs. The HUMCDDANF_{—}1P4 (SEQ ID NO:15) variant protein is secreted.

Variant protein HUMCDDANF_{—}1_P4 (SEQ ID NO:15) also shows polymorphism which is the result of non-silent SNPs (Single Nucleotide Polymorphisms) affecting the amino acid positions as is shown in Table 9, with the alternative amino acids listed. The last column of table 9 indicates whether the SNP is known or not; the presence of known SNPs in variant protein HUMCDDANF_{—}1_P4 (SEQ ID NO:15) sequence provides support for the deduced sequence of this variant protein according to the present invention.

TABLE 9
Amino acid mutations

| position of amino acid | Alternative | Previously |
|---|---|---|
| sequence affected by SNP | amino acid(s) | known SNP? |
| 27 | P-> | No |
| 32 | V -> M | Yes |
| 60 | P-> | No |
| 70 | A -> V | Yes |
| 76 | P -> L | No |
| 77 | L -> F | Yes |
| 124 | S-> | No |
| 126 | R -> Q | Yes |

The variant protein has the following domains, as determined using InterPro. The domains are described in Table 10:

TABLE 10
InterPro domain(s)

| Domain description | Analysis type | Position(s) on protein |
|---|---|---|
| Natriuretic peptide, | BlastProDom | 121-149 |
| atrial type | | |
| Natriuretic peptide | BlastProDom | 26-120, 150-150 |
| Natriuretic peptide | FPrintScan | 127-136, 136-145 |
| Natriuretic peptide, | FPrintScan | 11-29, 32-50, 51-69, 72-89, |
| atrial type | | 92-108, 109-127, 128-150 |
| Natriuretic peptide | HMMPfam | 43-146 |
| Natriuretic peptide | HMMSmart | 123-146 |
| Natriuretic peptide | ScanRegExp | 130-146 |

Variant protein HUMCDDANF_{—}1_P4 (SEQ ID NO:15) is encoded by (SEQ ID NO:1), for which the coding portion starts at position 196 and ends at position 801. The transcript also has the following SNPs as listed in Table 11 (given according to their position on the nucleotide sequence, with the alternative nucleic acid listed; the last column indicates whether the SNP is known or not; the presence of known SNPs in variant protein HUMCDDANF_{—}1_P4 (SEQ ID NO:15) sequence provides support for the deduced sequence of this variant protein according to the present invention).

TABLE 11
Nucleic acid SNPs

| SNP position(s) on | Alternative | Previously |
|---|---|---|
| nucleotide sequence | nucleic acid(s) | known SNP? |
| 123 | G -> C | No |
| 123 | G -> T | No |
| 132 | C-> | No |
| 276 | C-> | No |
| 289 | G -> A | Yes |
| 374 | C-> | No |
| 404 | C -> T | Yes |
| 422 | C -> T | No |
| 424 | C -> T | Yes |
| 567 | C-> | No |
| 572 | G -> A | Yes |
| 834 | A -> G | Yes |
| 855 | A -> G | Yes |
| 902 | G -> C | Yes |
| 994 | G -> A | Yes |
| 1007 | C -> T | Yes |
| 1080 | A -> G | Yes |
| 1147 | A -> G | Yes |
| 1174 | G -> A | Yes |
| 1206 | G -> A | Yes |
| 1378 | A -> G | Yes |
| 1747 | T -> C | Yes |
| 1820 | G -> T | Yes |
| 1834 | T -> C | Yes |
| 1841 | T -> C | Yes |
| 1872 | C -> T | Yes |
| 1950 | C -> G | No |
| 2031 | A -> C | Yes |

Variant protein HUMCDDANF_{—}1_P5 (SEQ ID NO:16) is encoded by transcript HUMCDDANF_{—}1_T4 (SEQ ID NO:2). An alignment of the variant protein to the known atrial natriuretic factor precursor is shown in FIG. 7. A Comparison between HUMCDDANF_{—}1_P5 (SEQ ID NO:16) and ANF_HUMAN is shown in FIG. 7A. A Comparison between HUMCDDANF_{—}1_P5 (SEQ ID NO:16) and NP_{—}006163 (SEQ ID NO:14) is shown in FIG. 7B. A brief description of the relationship of the variant protein HUMCDDANF_{—}1_P5 to each such aligned protein is as follows:

A. The isolated chimeric polypeptide designated HUMCDDANF_{—}1_P5 (SEQ ID NO:16) comprises a first amino acid sequence being at least 90% homologous to MSSFSTTTVSFLLLLAFQLLGQTRANPMYNAVSNADLMDFKNLLDHLEEKMPLE DEVVPPQVLSEPNEEAGAALSPLPEVPPWTGEVSPAQRDGGALGRGPWDSSDRS ALLKSKLRALLTAPRSLRRSSCFGGRMDRIGAQSGLGCNSFR corresponding to amino acids 1-150 of ANF_HUMAN (SEQ ID NO:13) or NP_{—}006163 (SEQ ID NO:14), which also corresponds to amino acids 1-150 of HUMCDDANF_{—}1_P5 (SEQ ID NO:16), and a second amino acid sequence being at least 70%, optionally at least 80%, preferably at least 85%, more preferably at least 90% and most preferably at least 95% homologous to a polypeptide having the sequence ELNWLRPLMEQPLLSLME (SEQ ID NO:40) corresponding to amino acids 151-168 of HUMCDDANF_{—}1_P5 (SEQ ID NO:16), wherein said first amino acid sequence and second amino acid sequence are contiguous and in a sequential order.

B. An isolated polypeptide designated an edge portion of HUMCDDANF_{—}1_P5 (SEQ ID NO:16) comprises an amino acid sequence being at least 70%, optionally at least about 80%, preferably at least about 85%, more preferably at least about 90% and most preferably at least about 95% homologous to the sequence ELNWLRPLMEQPLLSLME (SEQ ID NO:40) of HUMCDDANF_{—}1_P5 (SEQ ID NO:16).

C. A bridge portion of HUMCDDANF_{—}1_P5 (SEQ ID NO:16) comprises a polypeptide having a length “n”, wherein n is at least about 10 amino acids in length, optionally at least about 15 amino acids in length, preferably at least about 20 amino acids in length, more preferably at least about 25 amino acids in length and most preferably at least about 30 amino acids in length, wherein at least two amino acids comprise RE, having a structure as follows (numbering according to HUMCDDANF_{—}1_P5 (SEQ ID NO:16)): a sequence starting from any of amino acid numbers 150-x to 150; and ending at any of amino acid numbers 151+((n−2)−x), in which x varies from 0 to n−2.

The location of the variant protein was determined according to results from a number of different software programs and analyses, including analyses from SignalP and other specialized programs. HUMCDDANF_{—}1_P5 (SEQ ID NO:16) variant protein is a secreted protein.

Variant protein HUMCDDANF_{—}1_P5 (SEQ ID NO:16) also shows polymorphism which is the result of non-silent SNPs (Single Nucleotide Polymorphisms) affecting the amino acid positions as is shown in Table 12, with the alternative amino acids listed. The last column of Table 12 indicates whether the SNP is known or not; the presence of known SNPs in variant protein HUMCDDANF_{—}1_P5 (SEQ ID NO:16) sequence provides support for the deduced sequence of this variant protein according to the present invention).

TABLE 12
Amino acid mutations

| position of amino acid | Alternative | Previously |
|---|---|---|
| sequence affected by SNP | amino acid(s) | known SNP? |
| 27 | P-> | No |
| 32 | V -> M | Yes |
| 60 | P-> | No |
| 70 | A -> V | Yes |
| 76 | P -> L | No |
| 77 | L -> F | Yes |
| 124 | S-> | No |
| 126 | R -> Q | Yes |
| 156 | R -> * | Yes |
| 161 | Q -> R | Yes |

The variant protein has the following domains, as determined by using InterPro. The domains are described in Table 13:

TABLE 13
InterPro domain(s)

| Domain description | Analysis type | Position(s) on protein |
|---|---|---|
| Natriuretic peptide, | BlastProDom | 121-149 |
| atrial type | | |
| Natriuretic peptide | BlastProDom | 26-120, 150-151 |
| Natriuretic peptide | FPrintScan | 127-136, 136-145 |
| Natriuretic peptide, | FPrintScan | 11-29, 32-50, 51-69, 72-89, |
| atrial type | | 92-108, 109-127, 128-150 |
| Natriuretic peptide | HMMPfam | 43-146 |
| Natriuretic peptide | HMMSmart | 123-146 |
| Natriuretic peptide | ScanRegExp | 130-146 |

Variant protein HUMCDDANF_{—}1_P5 (SEQ ID NO:16) is encoded by HUMCDDANF_{—}1_T4 (SEQ ID NO:2), wherein the coding portion starts at position 196 and ends at position 699. The transcript also has the following SNPs as listed in Table 14 (given according to their position on the nucleotide sequence, with the alternative nucleic acid listed; the last column indicates whether the SNP is known or not; the presence of known SNPs in variant protein HUMCDDANF_{—}1_P5 (SEQ ID NO:16) sequence provides support for the deduced sequence of this variant protein according to the present invention).

TABLE 14
Nucleic acid SNPs

| SNP position(s) on | Alternative | Previously |
|---|---|---|
| nucleotide sequence | nucleic acid(s) | known SNP? |
| 123 | G -> C | No |
| 123 | G -> T | No |
| 132 | C-> | No |
| 276 | C-> | No |
| 289 | G -> A | Yes |
| 374 | C-> | No |
| 404 | C -> T | Yes |
| 422 | C -> T | No |
| 424 | C -> T | Yes |
| 567 | C-> | No |
| 572 | G -> A | Yes |
| 661 | C -> T | Yes |
| 677 | A -> G | Yes |
| 868 | G -> T | Yes |

### Example 3

### Validation of ANP Variants

The results described herein show that the ANP variants of the present invention are naturally expressed in hear tissues.

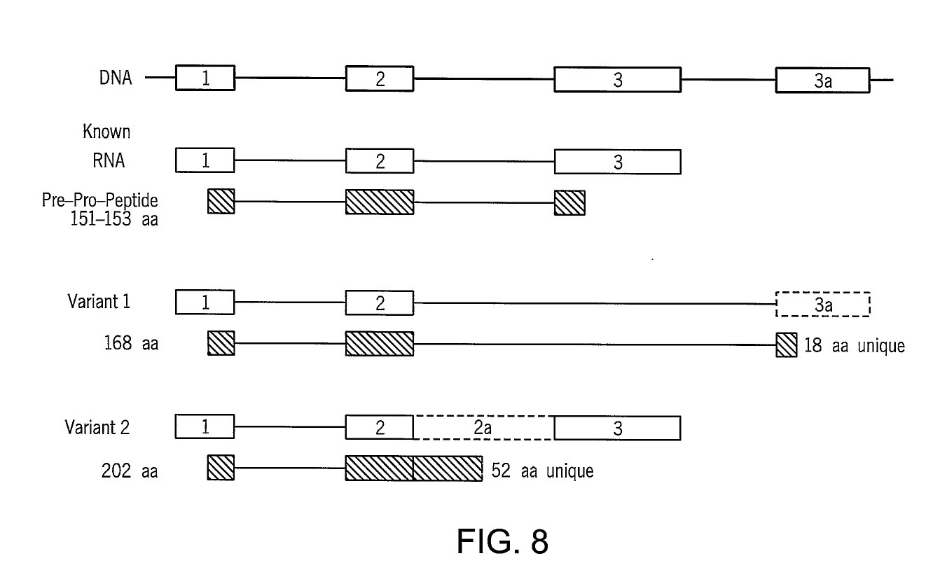

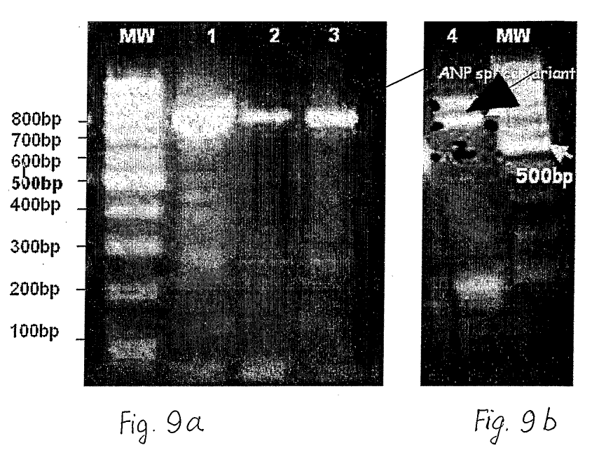

The ANP gene located on chromosome 1 has three exons and two introns as demonstrated in FIG. 8. One of the ANP transcript variants of the present invention, Variant 2 (SEQ ID NO:15), exhibits an intron retention between exon 2 and exon 3, and potentially encodes a protein with a novel C-terminus, as shown in FIG. 8. In this variant, the last Tyrosine is replaced by a unique tail of 52 amino acids. The presence of this alternative transcript (SEQ ID NO:1) was validated by RT-PCR performed on mRNA from fibrotic human heart tissue, as shown in FIG. 9.

FIG. 9a presents the products of RT-PCR performed on a mixture of total RNA extracted from the following tissues: line #1 from heart, bone, fibroblasts and brain tissues; line #2 from brain, kidney, heart, liver and testis tissues and line #3 from kidney, liver, ovary and blood. FIG. 9b presents the product of RT-PCR performed on total RNA extracted from a fibrotic heart. PCR bands representing ANP splice variant 2 (SEQ ID NO:1) are indicated by green arrows. The upper band in FIG. 9b is probably a product of a genomic contamination. MW is the molecular weight marker.

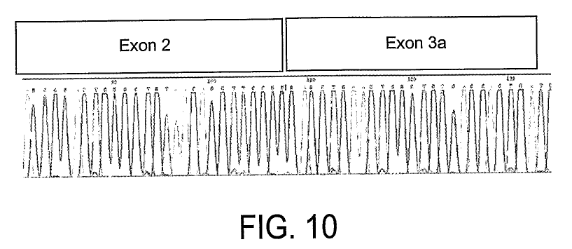

A second alternative ANP splice variant polynucleotide of the present invention, with an alternative 3rd exon, encodes a protein with a different novel C-terminus. In this ANP variant, Variant 1 (FIG. 8), the last Tyr is replaced by a unique tail of 18 amino acids. The expression of this alternative transcript was validated by RT-PCR performed on mRNA that was extracted from normal as well as failing heart tissues. Sequencing results for the ANP-variant 1 from failing heart is shown in FIG. 10.

FIG. 8 presents the gene structure of ANP as a bold-black chain, with introns as plain bold line and exons as boxes. The intron between exon 2 and 3 (intron 2) is shown as plain gray line, although it may be retained in the mature transcript. The various RNA transcripts are shown as plain-border boxes. The top RNA transcript depicts the known mRNA. The alternative variants 1 and 2 are shown below in that order as indicated. The corresponding pre-pro-peptides are depicted as boxes with upper left to lower right fill, below each transcript. The numbers indicate length of the proteins in amino-acids. There are two known human pre-pro-proteins created by a single nucleotide polymorphism (SNP), that differ by the absence or presence of a C-terminal Arginine-Arginine dipeptide, thus their length are 151 and 153 amino acids, respectively, as indicated for the Known Pre-Pro-Peptide. The unique amino acids of each pre-pro-protein ANP variant are depicted by a dashed frame and the length of the unique region is shown at its right side.

### Example 4

### ANP Peptides Alternative Processing

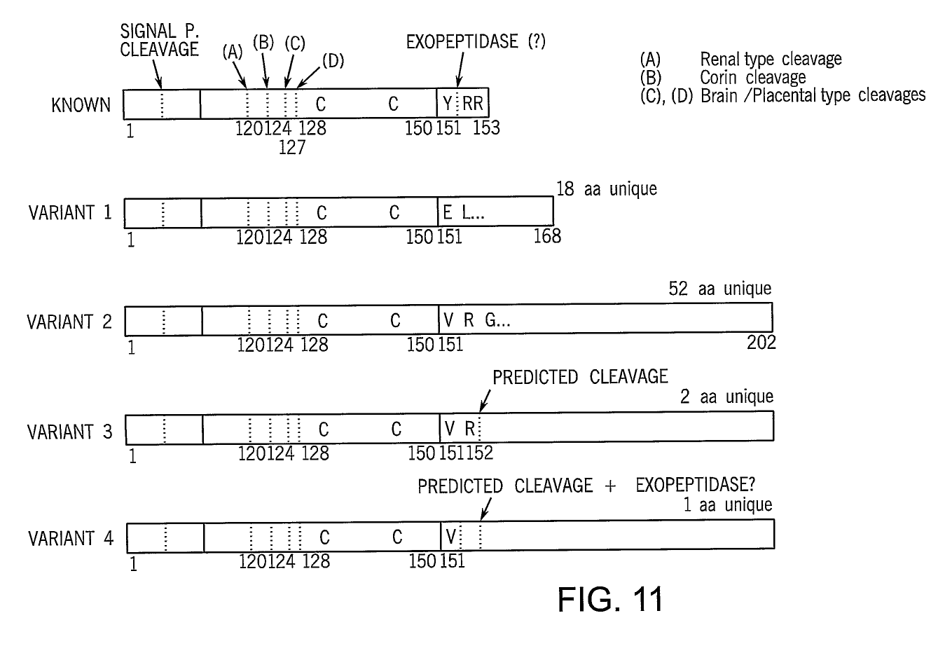

Four known alternative cleavage sites exist at the N-terminus of the active NAP peptide. In the atria, the pro-hormone is cleaved by corin at one site (R123-S124, numbering according to ANP precursor sequence ANF_HUMAN (SEQ ID NO:13)) to produce ANP. The cleavage product—ANP—is indicated as (B) in FIG. 11. In the kidney, the pro-peptide is cleaved four amino acids upstream (L119-T120; indicated as (A) in FIG. 11) to produce Urodilatin. In brain and placenta, cleavage occurs at sites L126-R127 and R127-R128; indicated in FIG. 11 as (C) and (D) respectively, resulting in 3-4 aa shorter peptides (ANP 4-28 and ANP 5-28) (Imura et al., Front Neuroendocrinol. 1992 13(3):217-49; Huang et al., Endocrinology. 1992 131(2):919-24.). Three ((A), (B) and (C)) out of the four alternative N-termini peptides retain similar activation of the receptor whereas the shortest cleavage product (D) shows some activation, although at a reduced level (Kangawa et al., 1984 Biochem Biophys Res Commun. 121(2):585-91.).

Using an algorithm as described in Example 1 hereinabove, (a classifier that was trained on experimental data extracted from the Swiss-Prot protein database), a previously unknown proteolytic cleavage site was detected within the C-terminal tail of the variant 2 protein (SEQ ID NO:15). This proteolysis results in ANP variant 3 as shown in FIG. 11. The resulting peptides include 3A (SEQ ID NO:29), 3B(SEQ ID NO:30), 3C (SEQ ID NO:31) and 3D (SEQ ID NO:32). In addition, as Exopeptidase E frequently cuts newly formed C-terminal arginine, ANP variant 4 can be synthesized, as shown in FIG. 11. The resulting peptides include 4A (SEQ ID NO:33), 4B (SEQ ID NO:34), 4C (SEQ ID NO:35) and 4D (SEQ ID NO:36).

Combining the different possibilities in RNA splicing and in N-terminal and C-terminal processing yields a large number of possible ANP peptide products, as is demonstrated in FIG. 11, presenting a cleavage map of ANP pre-pro-peptide variants. The pre-pro-peptides are depicted as boxes. Known regions are depicted by boxes between 1 to 150 (the numbers indicating the amino acid position (according to ANP precursor sequence ANF_HUMAN, SEQ ID NO:13). The unique region in each variant is depicted by a box with its length shown at right side. Semi-transparent areas represent parts that are cleaved out. Dotted lines indicate cleavage sites, the naming of which is noted by arrows and short descriptions (see also legend in insert). The amino acid at the C-terminal side of each cleavage site is denoted by its number (according to ANP precursor sequence ANF_HUMAN (SEQ ID NO:13)). Amino acid 127 (cleavage site (C)) is denoted only once, for clarity. ° C.'s represent conserved cysteines that form a disulfide bridge. One to three amino acids that reside immediately C-terminal to amino acid 150 (according to ANP precursor sequence ANF_HUMAN (SEQ ID NO:13)) are denoted in one-letter code.

### Example 5

### ANP Variants Peptide Synthesis

Peptides were synthesized by SPPS (solid phase peptide synthesis) method cleaved from the resin and purified by RP-HPLC. Then the ANP-45 (SEQ ID NO:22) and ANP-49 (SEQ ID NO:21) peptides were cyclized (disulfide bridge: 7-23 in ANP-45 and 11-27 in ANP-49) by air oxidation. The ANP-28 (SEQ ID NO:18) and ANP-32 (SEQ ID NO:17) peptides were cyclized by iodine oxidation (disulfide bridge: 7-23 in ANP-WT28 and 11-27 in ANP-32). All cyclic molecules were purified by RP-HPLC.

Peptide identity and the presence of disulfide bridge were verified by mass spectrometry.

Final purity of ANP-28 (SEQ ID NO:18), ANP-32 (SEQ ID NO:17), ANP-45 (SEQ ID NO:22) and ANP-49 (SEQ ID NO:21) was 96%, 98%, 98% and 93% respectively.

### Example 6

### Evaluation of the Agonistic Activity of the ANP Variants: Measurement of Cyclic GMP Accumulation After Activation of Membrane-Bound Guanylyl Cyclase

The agonist-like activity of the ANP variant peptides was evaluated by in vitro assays designed to measure cGMP accumulation after activation of membrane-bound guanylyl cyclase. Guanylyl cyclases (GC) are a family of enzymes (EC 4.6.1.2) that catalyse the formation of the second messenger cyclic GMP (cGMP) from GTP. Activation of GCs leads to an increase in the concentration of the intracellular messenger molecule cGMP. GCs are subdivided into soluble GCs and GCs that are membrane-bound and linked to a receptor. Activation occurs through binding of nitric oxide (NO) and peptide hormones, respectively. Six different isoforms of membrane-bound GC (GC-A, GC-B, GC-C, GC-D, GC-E and GC-F) are known. The guanylyl cyclase A (GC-A) isoform acts as the receptor for the natriuretic peptides ANP and BNP.

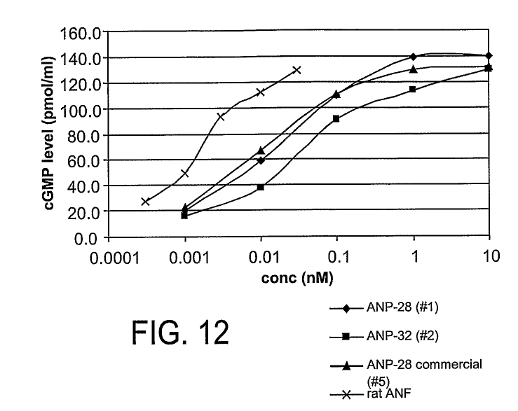

As ANP is known to stimulate GC-A, the effect of known and variant ANP peptides on guanlyl cyclase was examined. The procedure employed was essentially as described in Glover V, et al., 1995 Life Sci. 57(22): 2073-2079; Nashida T, Imai A and Shimomura H (2000), Mol Cell Biochem. 208(1-2): 27-35. Briefly, membrane-bound guanylyl cyclase isolated from the lung of Wistar rats was used. Test compound and/or vehicle were incubated with 20 mg/ml enzyme in Tris buffer pH 7.6 at 37° C. for 20 minutes. The reaction was terminated by addition of 1 N HCl and the supernatant evaluated for cGMP level by EIA. Enzyme activity was determined spectrophotometrically by measuring the formation of cGMP. Test compound-induced cGMP increase of 50 percent (50%) or more relative to the response observed with 1 mM ANF (rat) control indicates possible agonist activity. Compounds were screened in triplicate (n−3) at 10, 1, 0.1, 0.01 and 0.001 mM. Rat ANF (rat known ANP) was used as a reference compound. Due to species specificity, the measured activity of the reference human peptide ANP (ANP-28, SEQ ID NO:18), is different from the rat ANF activity in this assay. However, ANP-28 (SEQ ID NO:18) synthesized in-house has similar activity to human ANP from a commercial source (as shown in FIG. 12).

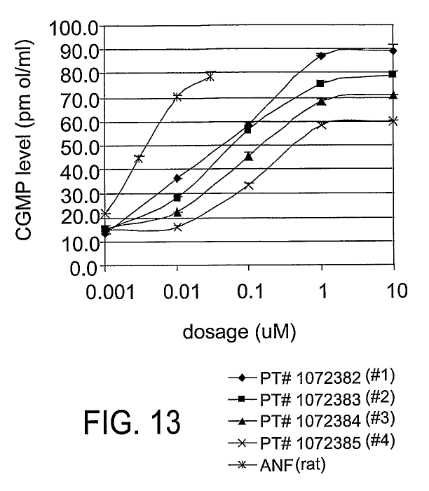

Thus, the activity of the ANP variant peptides, ANP-45 (SEQ ID NO:22) and ANP-49 (SEQ ID NO:21), was compared to that of the human ANP-28 (SEQ ID NO:18) and ANP-32 (SEQ ID NO:17) peptides (derived from known human ANP) which were synthesized in house, as described above. FIG. 13 presents response curves demonstrating the activity of wild type ANP peptides #1 (CGD-1, PT# 1072382, ANP28, SEQ ID NO:18) and #2 (CGD-2, PT# 1072383, ANP32, SEQ ID NO:17) and agonist-like activity of the variant ANP peptides peptide #3 (CGD-3, PT# 1072384, ANP45, SEQ ID NO:22) and #4 (CGD-4, PT# 1072385, ANP49, SEQ ID NO:21), measured as accumulated cGMP in isolated rat lung membranes. All four compounds showed increased binding with respective EC50 values for CGD-1, CGD-2, CGD-3 and CGD-4, of 59, 49.2, 86.3, and 138 nM.

The results presented in FIG. 13 demonstrate that ANP variants of the present invention are active ANP agonists that can induce cGMP accumulation in this experimental system at a level comparable to the known control molecules (ANP 45 (SEQ ID NO:22) and ANP 49 (SEQ ID NO:21) relative to ANP 28 (ANP, SEQ ID NO:18) and ANP 32 (Urodilatin, SEQ ID NO:17) respectively).

### Example 7

### Assessment of Renal and Cardiovascular Effects of ANP Variants

Renal and cardiovascular effects of ANP variants of the present invention, ANP45 (SEQ ID NO:22) and ANP49 (SEQ ID NO:21), were evaluated in parallel to the effects of ANP28 (SEQ ID NO:18), Urodilatin (ANP-32, SEQ ID NO:17) and Carperitide (NP-1). The assay was performed with anesthetized spontaneously hypertensive rats (SHR) following intravenous administration of the various ANP-proteins by a 30-min infusion.

Male Wistar-Okamoto derived spontaneously hypertensive rats (SHR) provided by National Laboratory Animal Center were used. Space allocation for 6 rats was 45×23×15 cm. All animals were maintained in a controlled temperature (23° C.-24° C.) and humidity (60%-70%) environment with 12 hours light dark cycles for at least one week in MDS Pharma Services—Taiwan Laboratory prior to use. Free access to standard lab chow (Laboratory Rodent Diet MF-18; Oriental Yeast Co., Ltd. Japan) and RO water was granted. All aspects of this work including housing, experimentation and disposal of animals were performed in general accordance with the Guide for the Care and Use of Laboratory Animals (National Academy Press, Washington, D.C., 1996).

Groups of 5 male rats (SHR) weighing 250-300 g were employed. Rats were anaesthetized with Inactin (120 mg/kg i.p.). The left carotid artery, jugular vein, and bladder were cannulated with polyethylene (PE50) catheters. The arterial cannula was connected via a Statham (P 23×L) pressure transducer to a NEC/San-Ei Type 366 polygraph for direct mean arterial blood pressure measurements. Heart rate was derived from pulse pressure signals and monitored on a Data Acquisition and Analytic System (Power Lab 8/SP, AD Instruments, Australia). The body temperature was kept at 37° C. using a thermostat-controlled hot plate. The animals were primed with 1 ml 2.5% dextrose+0.45% NaCl intravenously, followed by continuous infusion of the same solution at a rate of 2.4 ml/hr with an infusion pump throughout the experiments. After an equilibration period of 30 min, a 30-min control urine sample was collected. Test compounds were dissolved in 2.5% dextrose+0.45% NaCl and delivered by infusion for 30 min at 2.4 ml/hr. The treatment groups included: vehicle control (no compound); ANP-28, ANP-32, NP-1 (1 μg/kg/min); ANP-45, and ANP-49 (1 and 3 μg/kg/min). The infusion rate was 2.4 ml/hr. Urine samples were collected through the catheter during infusion of peptides and in the two 30-min post-peptide periods. Urinary Na⁺ concentration was measured with an atomic absorption spectrometer. All values represent mean±SEM and Dunnett's test was applied for comparison between corresponding time points for each drug-treated and control groups. Differences were considered significant at P<0.05.

The results of renal and cardiovascular effects of ANP45 (SEQ ID NO:22) and ANP49 (SEQ ID NO:21) as compared to these of ANP28 (SEQ ID NO:18), Urodilatin (SEQ ID NO:17) and Carperitide in anesthetized SHR following intravenous administration by a 30-min infusion are summarized below:

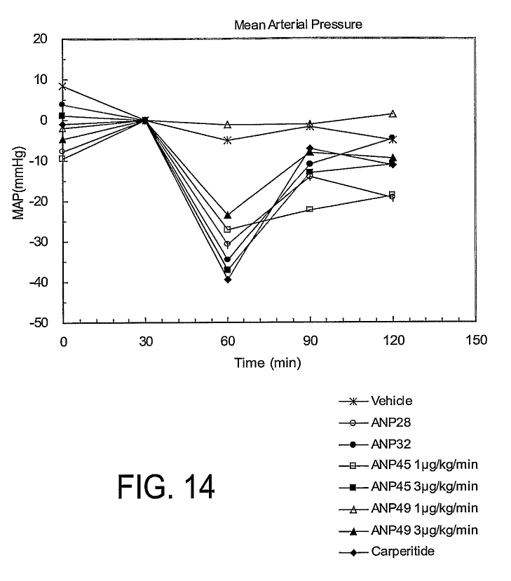

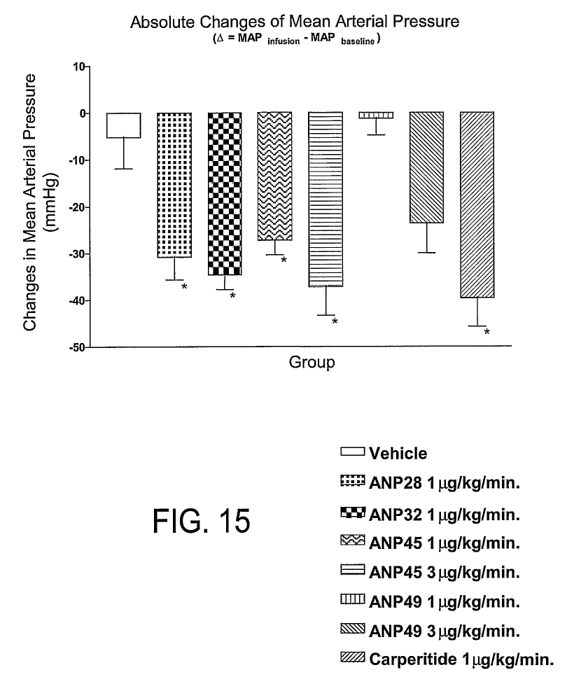

Mean arterial pressure (BP), as demonstrated in FIGS. 14-15: Baseline BP values were in general stable over time. The hypotensive response to ANP28, Urodilatin and Carperitide at 1 μg/kg/min during 30 min (1 μg/kg/min×30) was robust and similar in magnitude (−31-40 mmHg). ANP45 (SEQ ID NO:22) showed significant BP effect at 1 and 3 μg/kg/min×30 (−28 and −37 mmHg). ANP49 (SEQ ID NO:21) had no effect at 1 μg/kg/min×30 (−1 mmHg), but at 3 μg/kg/min×30 had a moderate effect on the BP (−23 mmHg). In general, BP values recovered rapidly after removal of the infusion for all the peptides tested. The peptides did not appear to differ in duration of action. FIG. 14 shows the results of mean arterial pressure (MAP), at the different time points. All values were normalized to the MAP value at the time of initiation of peptide infusion (30 min). The values represent the mean of the normalized values of 5 animals for each group at the 0, 30, 60, 90 and 120 minutes time points. FIG. 15 shows the absolute changes of mean arterial pressure, calculated as delta between MAP-infusion (60 min time point) and MAP-baseline (30 min time point). The values represent the mean±SEM of 5 animals for each group. *p<0.05 vs vehicle control.

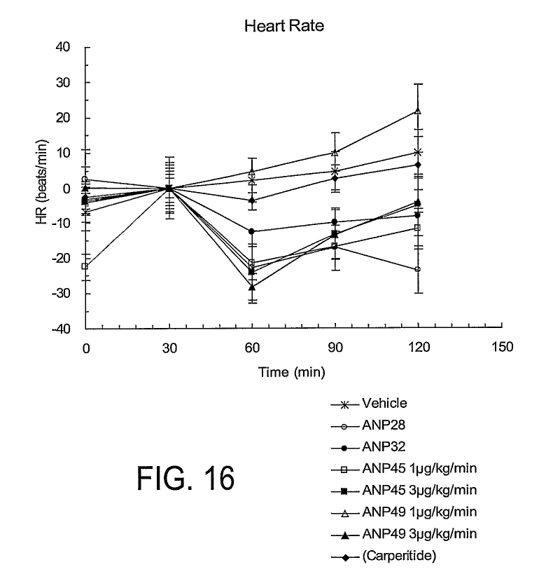

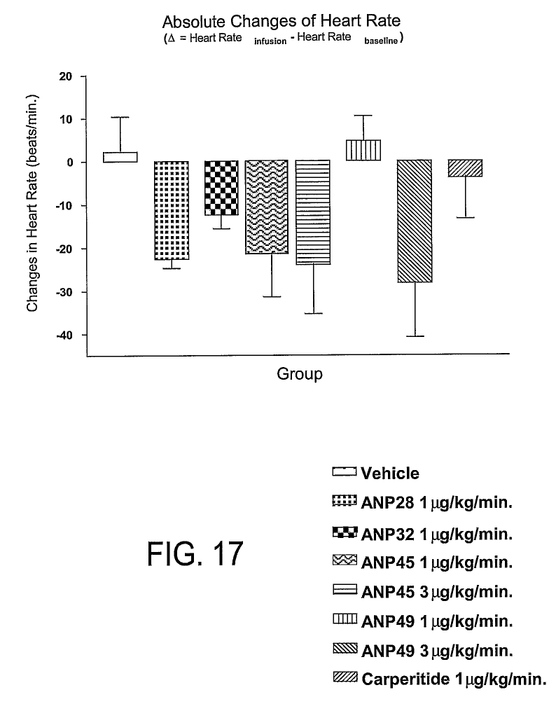

Heart rate (HR), as demonstrated in FIGS. 16-17: The hypotensive response to ANP28 (SEQ ID NO:18), Urodilatin (SEQ ID NO:17), ANP45 (SEQ ID NO:22) and ANP49 (SEQ ID NO:21) were accompanied by a decrease in heart rate, whereas the changes in HR in response to Carperitide were less pronounced. The results of the heart rate at the different time points are shown in FIG. 16. All values are expressed as delta between the measurement at the relevant time point and the baseline heart rate (30 min). The values represent the mean of 5 animals for each group at the 0, 30, 60, 90 and 120 minutes time points. *p<0.05 vs vehicle control. FIG. 17 shows the absolute changes of the heart rate, calculated as delta between the heart rate after infusion and the baseline heart rate. The values represent the mean±SEM of 5 animals for each group. All values (except vehicle control group) do not have significant variance vs. vehicle control.

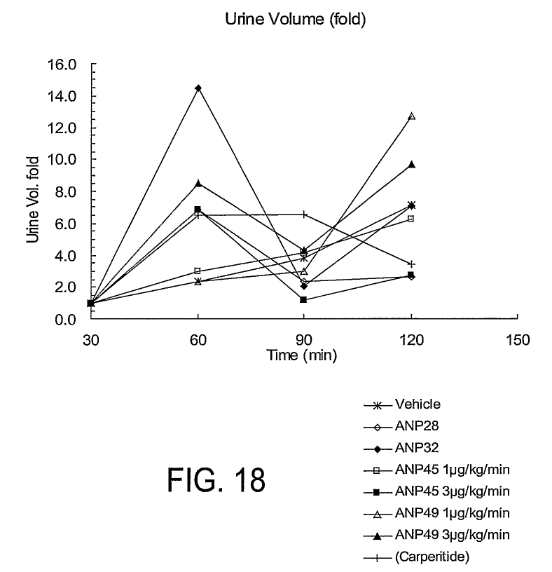

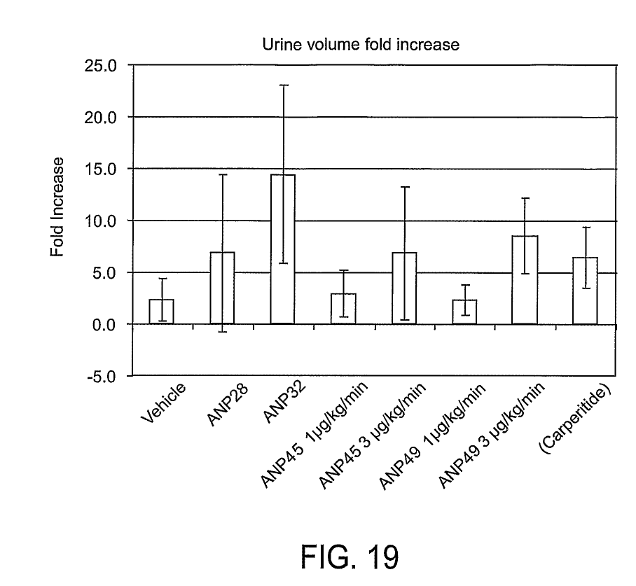

Urine volume, as demonstrated in FIGS. 18-19: Baseline urine volume values were very variable, therefore values are shown as fold increase of urine volume at each time point relative to the baseline value. Nonetheless, Urodilatin at 1 μg/kg/min×30 was associated with significant diuresis, and Carperitide and ANP28 at 1 μg/kg/min×30 were associated with significant but milder effect. In comparison, ANP45 (SEQ ID NO:22) caused a dose-dependent increase in urine volume, with the higher dose of 3 μg/kg/min×30 showing pronounced diuresis, similar in magnitude to that of Carperitide. ANP49 at 3 μg/kg/min×30 (SEQ ID NO:21) was associated with an even higher increase in urine volume relative to Carperitide only but such effect was not observed at the lower dose. The results of the urine volume analysis are shown in FIG. 18. The values represent the mean±SEM fold increase of 5 animals for each group at the 30-60 min, 60-90 min and 90-120 min time intervals. The reduction and variability in urine volume at 60-90 min and 90-120 min time intervals may be due to technical problems such as dehydration of the animals. FIG. 19 shows the relative changes in the urine volume, calculated as ratio between the urine volume after infusion and the baseline urine volume. The values represent the mean±SEM of 5 animals for each group.

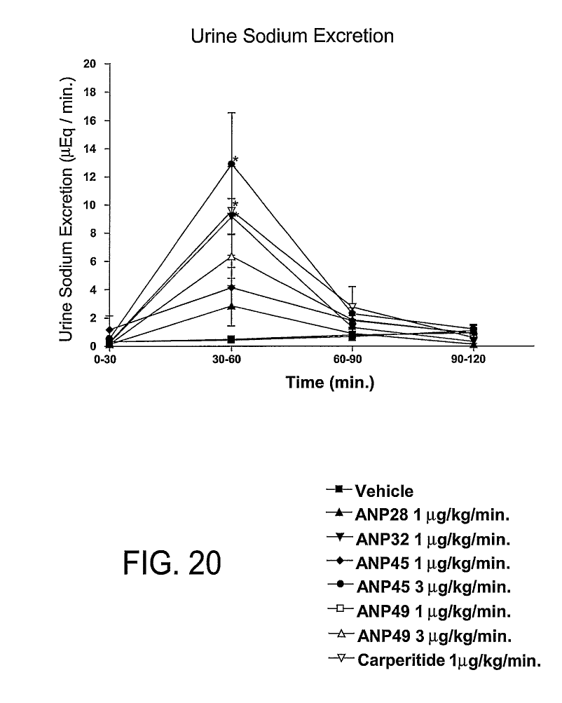

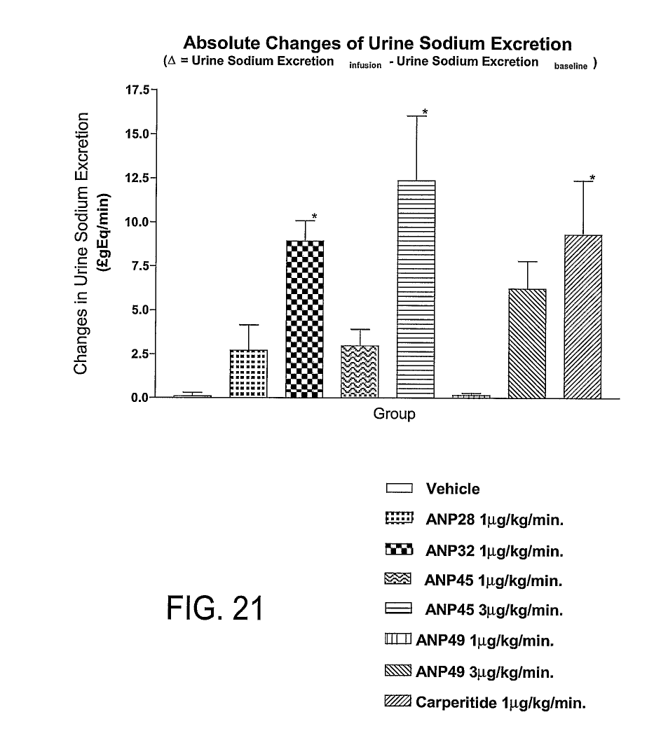

Natriuresis, as demonstrated in FIGS. 20-21: In general, the natriuretic response to the various peptides was in parallel to changes in urine volume. ANP45 (SEQ ID NO:22) caused a dose-dependent increase in urinary sodium excretion, showing greater potency than ANP49 (SEQ ID NO:21), Urodilatin and Carperitide.

The results of the urine sodium excretion analysis are shown in FIG. 20. The values represent the mean±SEM of 5 animals for each group at the 0-30 min, 30-60 min, 60-90 min and 90-120 min time intervals. *p<0.05 vs vehicle control. FIG. 21 shows the absolute changes in the urine sodium excretion, calculated as delta between the urine sodium excretion after infusion and the baseline urine sodium excretion. The values represent the mean±SEM of 5 animals for each group. *p<0.05 vs vehicle control.

It is concluded that ANP45 (1 and 3 μg/kg/min×30) caused a dose-dependent diuresis and natriuresis in association with hypotension and bradycardia, showing greater potency than ANP49 in most parameters except for urine volume fold increase, in which ANP49 at 3 μg/kg/min×30 dose showed a stronger effect. The duration of renal and cardiovascular action of the peptides at the 3 μg/kg/min x 30 dose appeared similar to Urodilatin, Carperitide and ANP28 in SHR. It should be noted that ANP 45 (SEQ ID NO:22) is larger than its reference wt peptide, ANP28 (SEQ ID NO:18), with a molecular weight ratio of 1.58 between them. Similarly, the molecular weight ratio between ANP 49 (SEQ ID NO:21) and ANP 32 (SEQ ID NO:17) is 1.66. Therefore, the difference in dose between 1 μg/kg/min×30 of ANP 28 (SEQ ID NO:18) and ANP32 (SEQ ID NO:17) and 3 μg/kg/min×30 for ANP45 (SEQ ID NO:22) and ANP49 (SEQ ID NO:21) is only, 1.89 and 1.8 fold, respectively.

In conclusion, the ANP variants of this invention showed significant effects on hemodynamic and renal parameters in vivo.

### Example 8

### Evolving CHF Produced by Rapid Ventricular Pacing in Dogs

### Experimental Protocol

Experiments are performed on six male mongrel dogs weighing between 18 and 23 kg that are fed normal dog chow and allowed free access to tap water. Experimental CHF is produced by 8 days of rapid ventricular pacing. This experimental model consistently produces a state of low-output cardiac failure with a constellation of cardiovascular, renal, and neurohumoral abnormalities characteristic of chronic primary myocardial failure.

Programmable cardiac pacemakers are implanted before acute interventions. Under pentobarbital anesthesia (30 mg/kg) and via a left thoracotomy and pericardiectomy, the heart is exposed and a screw-in epicardial pacemaker lead implanted into the right ventricle. The pacemaker lead is connected to a pulse generator implanted subcutaneously in the chest. The pericardium is sutured closed, and the parietal pleura and skin are closed in layers. The dogs are allowed to recover over a 3-day period, during which they receive prophylactic antibiotic treatment. Four to six days after pacemaker implantation, after an overnight fast, dogs are briefly anesthetized with sodium pentothal (15 mg/kg) to allow percutaneous placement of a flow-directed, balloon-tipped pulmonary artery catheter (model 93A-131-7F, American Edwards Laboratories, AHS del Caribe, Inc) via an external jugular vein. At the same time, a second balloon-tipped catheter is inserted into the urinary bladder. An intravenous infusion of 0.9% saline is initiated, and the animals are placed in a minimally restraining sling and allowed to regain consciousness and equilibrate over 90 to 120 minutes.

At the conclusion of this equilibration period, the acute experimental protocol is performed with the animals in the conscious state. The bladder is emptied before and at the end of all urinary clearances. In addition, at the midpoint of these and subsequent clearance periods, cardiac hemodynamics is measured and a 20-mL blood sample is withdrawn from the jugular vein. Two 30-minute baseline urinary clearances are performed. After baseline clearances, ANP variants are administered intravenously over 1 minute. A 30-minute urinary clearance is performed immediately after the ANP administration. The pulmonary artery and urinary catheters are then removed, and the dogs are returned to metabolic cages.

One day after exogenous ANP or ANP variants administration, the first of seven consecutive 24-hour urinary clearances is performed; all clearances are performed with the animals in the conscious state. The dogs' bladders are emptied with a urinary catheter at the beginning and end of each of these clearance periods. The urine collection devices located beneath the metabolic cages are designed to assure prompt freezing of voided urine until the time of analysis. Blood samples are obtained from a foreleg vein at the conclusion of each of these 24-hour clearance periods. After the first 24-hour clearance is completed, the pacemaker is programmed to 250 beats per minute, and pacing continued for the remainder of the experimental protocol. A series of six additional 24-hour urinary clearances are performed during the evolution of experimental CHF.

After 6 days of pacing and an overnight fast, dogs in each group are again briefly anesthetized with sodium pentothal (7 to 10 mg/kg) for placement of pulmonary artery and bladder catheters. The acute experimental protocol is performed with the animals in the conscious state in a manner identical to that described above for the baseline phase. At the conclusion of the protocol, catheters are removed, and the dogs returned to their cages.

### Analysis

Cardiac hemodynamic parameters measured during each 30-minute clearance period include right atrial pressure, pulmonary capillary wedge pressure, and cardiac output. Cardiac output is determined by thermodilution (cardiac output model 9510-A computer, American Edwards Laboratories), measured in quadruplicate, and averaged during each clearance period. All voided urine is collected on ice, measured using a graduated cylinder, and aliquots are made for measurement of sodium, creatinine, and cGMP. Urine samples for sodium and creatinine determination are refrigerated until analysis. Urine samples for cGMP determination are heated to 90° C. and kept at −20° C. until analysis. Net renal generation of cGMP is determined using the formula: Net renal generation of cGMP=(urinary cGMPxurine flow rate)-(plasma cGMPxcreatinine clearance).

Venous blood for sodium and creatinine determination is collected in heparinized tubes, placed on ice, and centrifuged at 2500 rpm at 4° C. After centrifugation, plasma is decanted and refrigerated until it analyzed. Plasma and urinary sodium concentrations are measured using ion-selective electrodes (Beckman Instruments). Glomerular filtration rate (GFR) is determined by creatinine clearance. Plasma and urine creatinine concentrations are measured by the Jaffe reaction (Beckman Instruments). Venous blood samples for hormone and cGMP analysis are placed in EDTA tubes, immediately placed on ice, and centrifuged at 2500 rpm at 4° C. Extracted venous plasma levels of ANP are measured by radioimmunoassay. Plasma renin activity is determined by radioimmunoassay. Plasma samples for cGMP are extracted with ethanol, and plasma and urinary cGMP are measured by radioimmunoassay.

All data are presented as mean±SEM. Comparisons between pre-CHF and subsequent 24-hour clearances are analyzed by repeated-measures ANOVA followed by Dunnett's t test when appropriate. Exogenous ANP responses in baseline CHF phases are compared by paired Student's t test.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-37. (canceled)

38. An isolated polynucleotide designated HUMCDDANF_{—}1_T4, coding for an ANP variant comprising the amino acid sequence ELNWLRPLMEQPLLSLME (amino acids 151-168 of SEQ ID NO:16).

39. The isolated polynucleotide according to claim 38, comprising a nucleic acid sequence coding for an ANP variant comprising the amino acid sequence set forth in SEQ ID NO:16.

40. The isolated polynucleotide of claim 38, comprising a nucleic acid sequence as set forth in SEQ ID NO:2.

41. The isolated polynucleotide according to claim 38, comprising a nucleic acid sequence at least about 95% homologous to SEQ ID NO:2.

42. An isolated ANP variant polypeptide comprising a first amino acid sequence at least 90% homologous to amino acids 1-150 of ANF_HUMAN (SEQ ID NO:13), which also corresponds to amino acids 1-150 of HUMCDDANF_{—}1_P5 (SEQ ID NO:16), and a second amino acid sequence at least 70% homologous to a polypeptide having an amino acid sequence as set forth in SEQ ID NO:40, corresponding to amino acids 151-168 of HUMCDDANF_{—}1_P5 (SEQ ID NO:16), wherein the first amino acid sequence and the second amino acid sequence are contiguous and in sequential order.

43. The polypeptide according to claim 42, wherein the second amino acid sequence is at least about 80% homologous to SEQ ID NO:40.

44. The polypeptide according to claim 42, wherein the second amino acid sequence is at least about 85% homologous to SEQ ID NO:40.

45. The polypeptide according to claim 42, wherein the second amino acid sequence is at least about 90% homologous to SEQ ID NO:40.

46. The polypeptide according to claim 42, wherein the second amino acid sequence is at least about 95% homologous to SEQ ID NO:40.

47. The isolated polypeptide according to claim 42, comprising an amino acid sequence at least about 95% homologous to SEQ ID NO:16 (HUMCDDANF_{—}1_P5).

48. The isolated polypeptide according to claim 47, comprising the amino acid sequence as set forth in SEQ ID NO:16.

49. An isolated ANP variant polypeptide comprising an amino acid sequence as set forth in any one of SEQ ID NO:22 (ANP45) and SEQ ID NO:21 (ANP49).

50. An isolated ANP variant polypeptide comprising an amino acid sequence as set forth in any one of SEQ ID NO:26 (ANP79) and SEQ ID NO: 25 (ANP83).

51. An isolated ANP variant polypeptide comprising an amino acid sequence as set forth in any one of SEQ ID NOs:23-24 and 27-36.

52. An isolated polypeptide designated a tail portion of HUMCDDANF_{—}1_P5 (SEQ ID NO:16), consisting of an amino acid sequence at least 70% homologous to a polypeptide having a sequence as set forth in SEQ ID NO:40.

53. The polypeptide according to claim 52, consisting of an amino acid sequence at least about 80% homologous to SEQ ID NO:40.

54. The polypeptide according to claim 52, consisting of an amino acid sequence at least about 85% homologous to SEQ ID NO:40.

55. The polypeptide according to claim 52, consisting of an amino acid sequence at least about 90% homologous to the sequence as set forth in SEQ ID NO:40.

56. The polypeptide according to claim 52, consisting of an amino acid sequence at least about 95% homologous to SEQ ID NO:40.

57. An isolated polynucleotide coding for a tail portion of HUMCDDANF_{—}1_P5 having an amino acid sequence as set forth in SEQ ID NO:40.

58. An expression vector comprising the polynucleotide sequence according to claim 38.

59. A host cell comprising the vector according to claim 58.

60. A process for producing a polypeptide comprising: (i) culturing the host cell according to claim 59 under conditions suitable to produce the polypeptide encoded by said polynucleotide and; (ii) recovering said polypeptide.

61. A pharmaceutical composition comprising as an active ingredient a polynucleotide sequence according to claim 38, further comprising a pharmaceutically acceptable diluent or carrier.

62. A pharmaceutical composition comprising as an active ingredient an expression vector according to claim 58, further comprising a pharmaceutically acceptable diluent or carrier.

63. A pharmaceutical composition comprising as an active ingredient a host cell according to claim 59, further comprising a pharmaceutically acceptable diluent or carrier.

64. A pharmaceutical composition comprising as an active ingredient a polypeptide according to claim 42, further comprising a pharmaceutically acceptable diluent or carrier.

65. A pharmaceutical composition comprising as an active ingredient a polypeptide according to claim 49.

66. A pharmaceutical composition comprising as an active ingredient a polypeptide according to claim 50.

67. A pharmaceutical composition comprising as an active ingredient a polypeptide according to claim 51.

68. A pharmaceutical composition comprising as an active ingredient a polypeptide according to claim 52.

69. A method for preventing, treating or ameliorating an ANP-related disease or disorder, comprising administering to a subject in need thereof a pharmaceutical composition according to claim 61.

70. The method according to claim 69, wherein the ANP-related disease is selected from the group consisting of cardiovascular diseases, such as acute or chronic heart failure, stroke, ischemic stroke, hemorrhagic stroke or transient ischemic attacks, myocardial infarction, stable or unstable angina pectoris, arrhythmias, valvular diseases, conditions that cause atrial and or ventricular wall volume overload, systemic arterial hypertension, pulmonary hypertension, pulmonary embolism, respiratory distress syndrome, conditions in which vasorelaxation or vasodilatation is efficacious, conditions in which diuresis is efficacious, asthma, obstructive lung disease, COPD, cardiomyopathy, myocarditis, congestive heart failure, CVS diseases, atrial or ventricular septal defects.

71. The method according to claim 69, wherein the ANP-related disease is selected from the group consisting of renal function relate disorders, such as diuresis, natriuresis, nephrotic syndrome, hepatic cirrhosis, pulmonary disease, acute or chronic renal failure, chronic kidney failure with residual kidney functions, cardiac insufficiency with oedematosis or sodium retention, oliguric renal failure, blood pressure disregulation, ascites in chronic liver diseases, vasopressin disregulation, posterior pituitary malfunction or chronic renal insufficiency.

72. The method according to claim 69, wherein the ANP-related disease is selected from the group consisting of cancer, such as brain cancer, breast adenocarcinoma, colon adenocarcinomas, prostate adenocarcinoma or lung cancers such as small cell or squamous cell lung adenocarcinoma.

73. The method according to claim 69, wherein the ANP-related disease is selected from the group consisting of fibrotic, inflammatory or allergic diseases such as myointimal proliferation in atherosclerosis, restenosis induced by angioplasty or vascular reconstructive surgery, glomerulonephritis or glomerulosclerosis.

74. The method according to claim 69, wherein the ANP-related disease is selected from the group consisting of keratoconjunctival failure, such as dry eye, corneal epithelial abrasion or corneal ulcer.

75. The method according to claim 69, wherein the ANP-related disease is selected from the group consisting of obesity, bone elongation, proliferation or survival of neurons.


SEQUENCE LISTING (41 sequences)

SEQ ID NO: 1 - DNA, 2049 bp, Homo sapiens
tttatcactgcaagtgacagaatggggagggttctgtctctcctgcgtgcttggagagctggggggctataaaaagaggcggcactgggcagctgggagacagggacagacgtaggccaagagaggggaaccagagaggaaccagaggggagagacagagcagcaagcagtggattgctccttgacgacgccagcatgagctccttctccaccaccaccgtgagcttcctccttttactggcattccagctcctaggtcagaccagagctaatcccatgtacaatgccgtgtccaacgcagacctgatggatttcaagaatttgctggaccatttggaagaaaagatgcctttagaagatgaggtcgtgcccccacaagtgctcagtgagccgaatgaagaagcgggggctgctctcagccccctccctgaggtgcctccctggaccggggaagtcagcccagcccagagagatggaggtgccctcgggcggggcccctgggactcctctgatcgatctgccctcctaaaaagcaagctgagggcgctgctcactgcccctcggagcctgcggagatccagctgcttcgggggcaggatggacaggattggagcccagagcggactgggctgtaacagcttccgggtaagaggaactggggatggaaatgggatgggatggacactactgggagacaccttcagcaggaaagggaccaatgcagaagctcattccctctcaagtttctgccccaacacccagagtgccccatgggtgtcaggacatgccatctattgtccttagctagtctgctgagaaaatgcttaaaaaaaaaaggggggggggctgggcacagtggctcacgcctgtaatcccagcactttgggaggccgaggcaggcggatcatgaggtcaagagatcaagactatcctggccaacatggtgaaaccccagctctactaaaaatacaaaaattagctgggtgtggtggcgggcacctgtactctcagctacttgggaggctgaggcaggagaatcacttgaacccaggaggcagaggttgcagtgagcagagatcacgccactgcagtccagcctaggtgatagagcgagactgtctcaaaaaaaaaaaaaaaaggccaggcacggtggctcacgcctgtaatcccagcgctttgggaggccaaggcgggtggatcacgaggtcaggagatggagaccatcctggctaacacggtgaaaccccgtctctactaaaaatacaaaaaattagccaggcgtggtggcaggcgcctgtaagtcctagctactccggaggctgaggcaggagaatggcgtgaacccgggaggcggagcttgcagtgagcagagatggcaccactgcactccagcctgggcgacagagcaagactccgtctcaaaaaaaaaaaaaaaaaaagcaactgccactagcactgggaaattaaaatattcatagagccaagttatctttgcatggctgattagcagttcatattcctccccagaattgcaagatcctgaagggcttaagtgaaatttactctgatgagtaacttgcttatcaattcatgaagctcagagggtcatcaggctggggtgggggccggtgggaagcaggtggtcagtaatcaagttcagaggatgggcacactcatacatgaagctgacttttccaggacagccaggtcaccaagccagatatgtctgtgttctctttgcagtactgaagataacagccagggaggacaagcagggctgggcctagggacagactgcaagaggctcctgtcccctggggtctctgctgcatttgtgtcatcttgttgccatggagttgtgatcatcccatctaagctgcagcttcctgtcaacacttctcacatcttatgctaactgtagataaagtggtttgatggtgacttcctcgcctctcccaccccatgcattaaattttaaggtagaacctcacctgttactgaaagtggtttgaaagtgaataaacttcagcaccatggacagaagacaaa

SEQ ID NO: 2 - DNA, 896 bp, Homo sapiens
tttatcactgcaagtgacagaatggggagggttctgtctctcctgcgtgcttggagagctggggggctataaaaagaggcggcactgggcagctgggagacagggacagacgtaggccaagagaggggaaccagagaggaaccagaggggagagacagagcagcaagcagtggattgctccttgacgacgccagcatgagctccttctccaccaccaccgtgagcttcctccttttactggcattccagctcctaggtcagaccagagctaatcccatgtacaatgccgtgtccaacgcagacctgatggatttcaagaatttgctggaccatttggaagaaaagatgcctttagaagatgaggtcgtgcccccacaagtgctcagtgagccgaatgaagaagcgggggctgctctcagccccctccctgaggtgcctccctggaccggggaagtcagcccagcccagagagatggaggtgccctcgggcggggcccctgggactcctctgatcgatctgccctcctaaaaagcaagctgagggcgctgctcactgcccctcggagcctgcggagatccagctgcttcgggggcaggatggacaggattggagcccagagcggactgggctgtaacagcttccgggagctgaactggctgcgaccactgatggaacagccacttctgtcactgatggaatgatgatttgaagcagatcagagacagaggccgctcttcttgcaaaaccaaaagcaagcgaacaaaaagcaatggccgatggcaacaacagaagctggccacgggtcttcacctctccagacccagcagcagtttcacttcttctggttggacatcggggcaatctgtgtgtggggcaacttcctaaacaccaggaa

SEQ ID NO: 3 - DNA, 219 bp, Homo sapiens
tttatcactgcaagtgacagaatggggagggttctgtctctcctgcgtgcttggagagctggggggctataaaaagaggcggcactgggcagctgggagacagggacagacgtaggccaagagaggggaaccagagaggaaccagaggggagagacagagcagcaagcagtggattgctccttgacgacgccagcatgagctccttctccaccaccacc

SEQ ID NO: 4 - DNA, 165 bp, Homo sapiens
aatttgctggaccatttggaagaaaagatgcctttagaagatgaggtcgtgcccccacaagtgctcagtgagccgaatgaagaagcgggggctgctctcagccccctccctgaggtgcctccctggaccggggaagtcagcccagcccagagagatggaggtgcc

SEQ ID NO: 5 - DNA, 139 bp, Homo sapiens
ctctgatcgatctgccctcctaaaaagcaagctgagggcgctgctcactgcccctcggagcctgcggagatccagctgcttcgggggcaggatggacaggattggagcccagagcggactgggctgtaacagcttccgg

SEQ ID NO: 6 - DNA, 1098 bp, Homo sapiens
gtaagaggaactggggatggaaatgggatgggatggacactactgggagacaccttcagcaggaaagggaccaatgcagaagctcattccctctcaagtttctgccccaacacccagagtgccccatgggtgtcaggacatgccatctattgtccttagctagtctgctgagaaaatgcttaaaaaaaaaaggggggggggctgggcacagtggctcacgcctgtaatcccagcactttgggaggccgaggcaggcggatcatgaggtcaagagatcaagactatcctggccaacatggtgaaaccccagctctactaaaaatacaaaaattagctgggtgtggtggcgggcacctgtactctcagctacttgggaggctgaggcaggagaatcacttgaacccaggaggcagaggttgcagtgagcagagatcacgccactgcagtccagcctaggtgatagagcgagactgtctcaaaaaaaaaaaaaaaaggccaggcacggtggctcacgcctgtaatcccagcgctttgggaggccaaggcgggtggatcacgaggtcaggagatggagaccatcctggctaacacggtgaaaccccgtctctactaaaaatacaaaaaattagccaggcgtggtggcaggcgcctgtaagtcctagctactccggaggctgaggcaggagaatggcgtgaacccgggaggcggagcttgcagtgagcagagatggcaccactgcactccagcctgggcgacagagcaagactccgtctcaaaaaaaaaaaaaaaaaaagcaactgccactagcactgggaaattaaaatattcatagagccaagttatctttgcatggctgattagcagttcatattcctccccagaattgcaagatcctgaagggcttaagtgaaatttactctgatgagtaacttgcttatcaattcatgaagctcagagggtcatcaggctggggtgggggccggtgggaagcaggtggtcagtaatcaagttcagaggatgggcacactcatacatgaagctgacttttccaggacagccaggtcaccaagccagatatgtctgtgttctctttgcag

SEQ ID NO: 7 - DNA, 260 bp, Homo sapiens
tactgaagataacagccagggaggacaagcagggctgggcctagggacagactgcaagaggctcctgtcccctggggtctctgctgcatttgtgtcatcttgttgccatggagttgtgatcatcccatctaagctgcagcttcctgtcaacacttctcacatcttatgctaactgtagataaagtggtttgatggtgacttcctcgcctctcccaccccatgcattaaattttaaggtagaacctcacctgttactgaaa

SEQ ID NO: 8 - DNA, 251 bp, Homo sapiens
gagctgaactggctgcgaccactgatggaacagccacttctgtcactgatggaatgatgatttgaagcagatcagagacagaggccgctcttcttgcaaaaccaaaagcaagcgaacaaaaagcaatggccgatggcaacaacagaagctggccacgggtcttcacctctccagacccagcagcagtttcacttcttctggttggacatcggggcaatctgtgtgtggggcaacttcctaaacaccaggaa

SEQ ID NO: 9 - DNA, 99 bp, Homo sapiens
gtgagcttcctccttttactggcattccagctcctaggtcagaccagagctaatcccatgtacaatgccgtgtccaacgcagacctgatggatttcaag

SEQ ID NO: 10 - DNA, 23 bp, Homo sapiens
ctcgggcggggcccctgggactc

SEQ ID NO: 11 - DNA, 12 bp, Homo sapiens
gtggtttgaaag

SEQ ID NO: 12 - DNA, 34 bp, Homo sapiens
tgaataaacttcagcaccatggacagaagacaaa

SEQ ID NO: 13 - protein, 153 aa, Homo sapiens [sequence omitted]

SEQ ID NO: 14 - protein, 151 aa, Homo sapiens
MSSFSTTTVSFLLLLAFQLLGQTRANPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRY

SEQ ID NO: 15 - protein, 202 aa, Homo sapiens
MSSFSTTTVSFLLLLAFQLLGQTRANPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRVRGTGDGNGMGWTLLGDTFSRKGTNAEAHSLSSFCPNTQSAPWVSGHAIYCP

SEQ ID NO: 16 - protein, 168 aa, Homo sapiens
MSSFSTTTVSFLLLLAFQLLGQTRANPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRELNWLRPLMEQPLLSLME

SEQ ID NO: 17 - protein, 32 aa, Homo sapiens
TAPRSLRRSSCFGGRMDRIGAQSGLGCNSFRY

SEQ ID NO: 18 - protein, 28 aa, Homo sapiens
SLRRSSCFGGRMDRIGAQSGLGCNSFRY

SEQ ID NO: 19 - protein, 25 aa, Homo sapiens
RSSCFGGRMDRIGAQSGLGCNSFRY

SEQ ID NO: 20 - protein, 24 aa, Homo sapiens
SSCFGGRMDRIGAQSGLGCNSFRY

SEQ ID NO: 21 - protein, 49 aa, Homo sapiens
TAPRSLRRSSCFGGRMDRIGAQSGLGCNSFRELNWLRPLMEQPLLSLME

SEQ ID NO: 22 - protein, 45 aa, Homo sapiens
SLRRSSCFGGRMDRIGAQSGLGCNSFRELNWLRPLMEQPLLSLME

SEQ ID NO: 23 - protein, 42 aa, Homo sapiens
RSSCFGGRMDRIGAQSGLGCNSFRELNWLRPLMEQPLLSLME

SEQ ID NO: 24 - protein, 41 aa, Homo sapiens
SSCFGGRMDRIGAQSGLGCNSFRELNWLRPLMEQPLLSLME

SEQ ID NO: 25 - protein, 83 aa, Homo sapiens
TAPRSLRRSSCFGGRMDRIGAQSGLGCNSFRVRGTGDGNGMGWTLLGDTFSRKGTNAEAHSLSSFCPNTQSAPWVSGHAIYCP

SEQ ID NO: 26 - protein, 79 aa, Homo sapiens
SLRRSSCFGGRMDRIGAQSGLGCNSFRVRGTGDGNGMGWTLLGDTFSRKGTNAEAHSLSSFCPNTQSAPWVSGHAIYCP

SEQ ID NO: 27 - protein, 76 aa, Homo sapiens
RSSCFGGRMDRIGAQSGLGCNSFRVRGTGDGNGMGWTLLGDTFSRKGTNAEAHSLSSFCPNTQSAPWVSGHAIYCP

SEQ ID NO: 28 - protein, 75 aa, Homo sapiens
SSCFGGRMDRIGAQSGLGCNSFRVRGTGDGNGMGWTLLGDTFSRKGTNAEAHSLSSFCPNTQSAPWVSGHAIYCP

SEQ ID NO: 29 - protein, 33 aa, Homo sapiens
TAPRSLRRSSCFGGRMDRIGAQSGLGCNSFRVR

SEQ ID NO: 30 - protein, 29 aa, Homo sapiens
SLRRSSCFGGRMDRIGAQSGLGCNSFRVR

SEQ ID NO: 31 - protein, 26 aa, Homo sapiens
RSSCFGGRMDRIGAQSGLGCNSFRVR

SEQ ID NO: 32 - protein, 25 aa, Homo sapiens
SSCFGGRMDRIGAQSGLGCNSFRVR

SEQ ID NO: 33 - protein, 32 aa, Homo sapiens
TAPRSLRRSSCFGGRMDRIGAQSGLGCNSFRV

SEQ ID NO: 34 - protein, 28 aa, Homo sapiens
SLRRSSCFGGRMDRIGAQSGLGCNSFRV

SEQ ID NO: 35 - protein, 25 aa, Homo sapiens
RSSCFGGRMDRIGAQSGLGCNSFRV

SEQ ID NO: 36 - protein, 24 aa, Homo sapiens
SSCFGGRMDRIGAQSGLGCNSFRV

SEQ ID NO: 37 - protein, 342 aa, Homo sapiens
MDPQTAPSRALLLLLFLHLAFLGGRSHPLGSLRRSSCFGGRMDRIGAQSGLGCNSFRVRGTGDGNGMGWTLLGDTFSRKGTNAEAHSLSSFCPNTQSAPWVSGHAIYCPFEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 346 aa, Homo sapiens [sequence omitted]

SEQ ID NO: 39 - protein, 52 aa, Artificial Sequence
  Synthetic peptide
VRGTGDGNGMGWTLLGDTFSRKGTNAEAHSLSSFCPNTQSAPWVSGHAIYCP

SEQ ID NO: 40 - protein, 18 aa, Artificial Sequence
  Synthetic peptide
ELNWLRPLMEQPLLSLME

SEQ ID NO: 41 - DNA, 845 bp, Homo sapiens
tggcgagggacagacgtaggccaagagaggggaaccagagaggaaccagaggggagagacagagcagcaagcagtggattgctccttgacgacgccagcatgagctccttctccaccaccaccgtgagcttcctccttttactggcattccagctcctaggtcagaccagagctaatcccatgtacaatgccgtgtccaacgcagacctgatggatttcaagaatttgctggaccatttggaagaaaagatgcctttagaagatgaggtcgtgcccccacaagtgctcagtgagccgaatgaagaagcgggggctgctctcagccccctccctgaggtgcctccctggaccggggaagtcagcccagcccagagagatggaggtgccctcgggcggggcccctgggactcctctgatcgatctgccctcctaaaaagcaagctgagggcgctgctcactgcccctcggagcctgcggagatccagctgcttcgggggcaggatggacaggattggagcccagagcggactgggctgtaacagcttccggtactgaagataacagccagggaggacaagcagggctgggcctagggacagactgcaagaggctcctgtcccctggggtctctgctgcatttgtgtcatcttgttgccatggagttgtgatcatcccatctaagctgcagcttcctgtcaacacttctcacatcttatgctaactgtagataaagtggtttgatggtgacttcctcgcctctcccaccccatgcattaaattttaaggtagaacctcacctgttactgaaagtggtttgaaagtgaataaacttcagcaccatggac